(19) United States
(12) Patent Application Publication
(10) Pub. No.: US 2016/0355896 A1
(43) Pub. Date: Dec. 8, 2016

(54) INCOATED RNA

(71) Applicant: AMPTEC GMBH, Hamburg (DE)

(72) Inventors: Guido Krupp, Gnutz (DE); Peter Scheinert, Heikendorf (DE); Holger Jeske, Stuttgart (DE); Christina Wege, Herrenberg (DE)

(21) Appl. No.: 15/117,857

(22) PCT Filed: Feb. 10, 2015
(86) PCT No.: PCT/EP2015/052803

§ 371 Date: Aug. 10, 2016

(30) Foreign Application Priority Data
Feb. 10, 2014 (EP) 14154518.6

(51) Int. Cl. C12Q 1/70; C12N 7/00

(52) U.S. Cl. CPC C12Q 1/701; C12N 7/00; C12N 2770/00023; C12Q 2600/166

(57) ABSTRACT

The present invention provides methods for the detection of RNA in a sample, comprising (a) mixing a specific amount of a rod-shaped virus-like particle with a sample, wherein the rod-shaped virus-like particle comprises a ribonucleic acid molecule and a viral coat, wherein: (i) the ribonucleic acid molecule comprises an origin-of-assembly sequence of a rod-shaped RNA virus and a heterologous sequence; and (ii) the viral coat comprises at least one type of coat protein of the rod-shaped RNA virus; (b) isolating RNA from the sample; and (c) detecting RNA comprising the heterologous sequence.

The present invention relates to methods for the detection of RNA in a sample, which methods comprise the use of a rod-shaped virus-like particle comprising a ribonucleic acid molecule and a viral coat, wherein the ribonucleic acid molecule comprises an origin-of-assembly sequence of a rod-shaped RNA virus and a heterologous sequence. Encapsidating heterologous RNA in rod-shaped virus-like particles in accordance with the present invention protects the RNA from degradation.

### TECHNICAL BACKGROUND

Rapid degradation of RNA by ubiquitous RNases represents an obstacle during experimental handling of RNA. Especially, the analysis of RNA obtained from biological samples is complicated by the presence of RNases endogenously present in the samples. A further problem in the analysis of RNA obtained from biological samples is the lack of stable internal controls. Absolute quantification of specific RNAs in biological samples is, however, an important part of diagnosis and treatment of, e.g., patients that have viral infections, such as infections with HIV or HCV. There is thus a need for RNA standards that improve quantification of RNA in a sample and assist to determine the extent of RNA degradation during experimental handling or storage of the sample.

The prior art proposed that RNA be protected from ribonucleases by encapsidation of the RNA using proteins of the bacteriophage MS2 (U.S. Pat. No. 5,677,124; US 2002/0192689). The encapsidated RNA was a hybrid of phage RNA and a foreign sequence that can be used as an RNA standard for the quantification of RNA in a sample. MS2 encapsidated RNAs are known as “Armored RNA”. The MS2 bacteriophage is an icosahedral structure composed of 180 coat proteins and a single A protein. The linear, ssRNA(+) genome is about 4 kb in size. Due to the icosahedral structure of the phage and the fixed size of about 27 nm in diameter, the length of the foreign RNA that may be inserted into the MS2 genome is limited to about 2 kb. Moreover, the natural hosts of MS2 are enterobacteria which can also be found in the intestines of mammals. Thus, use of the phage MS2 for diagnostics in a clinical setting bears a risk of reversion or crossing with the wild-type phage.

Further, it has been proposed to encapsidate RNA in a derivative of the Qbeta phage for use as RNA standard in molecular diagnostics (Villanova et al., 2007, J Clin Microbiol, p. 3555-63). Like MS2, Qbeta is a member of the Leviviridae viral family, with an icosahedral symmetry and e.g. E. coli as a natural host. Consequently, the Qbeta encapsidation has the same size-limitation and potential safety problems as the MS2 encapsidation.

Sleat et al. (1986, Virology, 155:299-308) attempted to incorporate RNAs into rod-shaped TMV particles. For that purpose RNA constructs were used comprising an origin-of-assembly (OAS) sequence of TMV with a length of approximately 440 nt. It was shown that the assembly of full TMV-like particles was limited to some of the chosen sequences. In none of the examples, the assembly process progressed to more than 1.5-1.6 kb of the RNA transcript. Other authors used very short OAS sequences. For example, Turner et al. (1988, J Mol Biol, 203:531-547) published that a TMV OAS sequence with a length of 75 nt was sufficient for TMV assembly. However, the inventors found that neither the very short, nor the long OAS constructs suggested by others in the art facilitate assembly of stable VLPs if the packaged RNA comprises a heterologous, non-TMV sequence. The constructs therefore lack the long-term stability that is required for commercial applications as an RNA standard. In consequence, these particles are unsuitable for use as a standard e.g. in commercial products and kits for detection of RNA.

There is thus a need in the art for RNA packaging systems which provide stable RNA and can universally be used to package heterologous RNA sequences.

### SUMMARY OF THE INVENTION

The present invention provides a method for the detection of RNA in a sample, comprising (a) mixing a specific amount of a rod-shaped virus-like particle with a sample, wherein the rod-shaped virus-like particle comprises a ribonucleic acid molecule and a viral coat, wherein: (i) the ribonucleic acid molecule comprises an origin-of-assembly sequence of a rod-shaped RNA virus and a heterologous sequence; and (ii) the viral coat comprises at least one type of coat protein of the rod-shaped RNA virus; (b) isolating RNA from the sample; and (c) detecting RNA comprising the heterologous sequence.

The present invention provides particularly advantageous virus-like particles (VLPs) encapsidating heterologous RNA that can serve as an internal RNA standard or as a positive control and methods using said VLPs. For example, the VLPs of the present invention can be used as a positive control to monitor sample processing or amplification steps. The VLPs can safely be used in clinical laboratories. The VLPs are prepared using a viral coat of a rod-shaped virus and RNA comprising an origin-of-assembly sequence of a rod-shaped virus. The protein coat of the VLP protects the RNA molecule from RNase digestion.

A significant advantage of the present invention resides in the fact that the size of the RNA within the VLP in essence is not subject to any size limitations because the length of the viral coat is automatically adapted to the length of the RNA molecule. The RNA may thus contain 200-100,000 nucleotides. The terms “nucleotide” (nt) and “ribonucleotide” are used interchangeably herein. It will be understood that the term “nucleotide” in the context of an RNA molecule refers to a ribonucleotide, while it may refer to a desoxyribonucleotide in the context of a DNA molecule.

A further advantage of the present invention resides in the fact that the VLPs of the invention are derived from viruses naturally infecting plant cells which enhances the safety of the VLP.

A further advantage of the embodiments of the invention is provided by the good stability of rod-shaped virus-like particles which is a mandatory prerequisite for their commercial use as a RNA standard and permits practicability in the storage and shipment of these products.

The present invention further provides the use of a rod-shaped virus-like particle as an internal RNA standard or as a positive control for RNA degradation and/or quantification, wherein the rod-shaped virus-like particle comprises a ribonucleic acid molecule and a viral coat, wherein:
(a) the ribonucleic acid molecule comprises an origin-of-assembly sequence of a rod-shaped RNA virus and a heterologous sequence; and
(b) the viral coat comprises at least one type of coat protein of the rod-shaped RNA virus.

In a further embodiment the present invention relates to the rod-shaped virus-like particle as such, which VLP comprises a ribonucleic acid molecule and a viral coat as defined above. The origin-of-assembly sequence comprised in the ribonucleic acid molecule of the rod-shaped virus-like particle of the invention has a length of from 150 to 300 nucleotides. Using an origin-of-assembly sequence having this length provides the necessary sequence and structural elements for efficient and universal (i.e. independent of the heterologous sequence) packaging and at the same time does not contain destabilizing elements.

In a related aspect, the invention provides a method for the manufacture of a rod-shaped virus-like particle comprising a ribonucleic acid molecule and a viral coat, comprising
(a) mixing a ribonucleic acid molecule and at least one type of coat protein of a rod-shaped virus, wherein the ribonucleic acid molecule comprises an origin-of-assembly sequence of a rod-shaped RNA virus and at least one heterologous sequence;
(b) heating the ribonucleic acid molecule and/or the at least one type of coat protein before step a) and/or heating the mixture at least once after step a) to a temperature between about 30° C. and about 100° C., preferably to about 70° C., more preferably to about 40° C.

According to the most preferred embodiment of the present invention, the rod-shaped virus like particle is derived from Tobacco Mosaic Virus (TMV), i.e. comprises TMV coat proteins and a TMV origin-of-assembly sequence (or OAS). Thus, the OAS is preferably a sequence having a length of up to 300 nucleotides comprising:
(A) SEQ ID NO:2; or
(B) a sequence having at least 85% identity to the sequence of SEQ ID NO:2; or
(C) a fragment of (A) or (B), wherein the fragment has a length of at least 150 nucleotides.

### DETAILED DESCRIPTION OF THE INVENTION

The method of the invention allows the detection of RNA in any sample, i.e. a sample of any origin. The sample may be any sample, such as a laboratory sample, a biological sample, or a clinical sample. In a preferred embodiment, the sample comprises ribonucleic acid (RNA). The sample may comprise cells or be cell-free. The sample may be solid or liquid. Preferably, the sample is a liquid, such as an aqueous solution.

A laboratory sample is a sample generated during experiments in a laboratory setting, such as a sample generated during chemical, biochemical or cellular synthesis of RNA. Suitable methods for the synthesis of RNA in cellular or cell-free systems are known to the person skilled in the art. Chemical synthesis of RNA can be carried out e.g. by solid phase synthesis. In solid phase synthesis, the building blocks of the ribonucleic acid oligonucleotide are immobilized e.g. on a bead and synthesized step by step in a reactant solution. Biochemical RNA synthesis is carried out e.g. by in vitro transcription of RNA, such as in the reaction described by Milburn et al., U.S. Pat. No. 5,256,555. During in vitro transcription of RNA, RNA molecules are synthesized by isolated and purified RNA polymerase enzymes using a template DNA that comprises an RNA polymerase promoter operably linked to a target sequence, a buffer system that includes DTT and suitable metal ions such as magnesium, manganese, cobalt etc., and ribonucleoside triphosphosphates (NTPs).

A biological sample is a sample derived from an organism, such as a bacterium, a fungus, a plant, or an animal, such as a vertebrate, e.g. a mammal. Further, the term “biological sample” comprises also samples derived from a cell or a collection of cells as cells have their origin in biological organisms. Thus, a biological sample may be a sample from a cell culture or a tissue culture.

A clinical sample is a sample obtained from an individual, wherein the individual is preferably a mammal, most preferably a human. For example, the clinical sample may be a sample selected from the group consisting of a body fluid, such as blood, urine, saliva, cerebrospinal fluid and the like; a tissue, such as an organ, skin, tumor and the like; and feces.

In step (a) of the above methods for the detection of RNA in a sample a specific amount of a rod-shaped virus-like particle (VLP) is mixed with a sample. A VLP resembles a virus, however, in the most preferred embodiments of the invention the VLP is replication-deficient. Thus, the VLP lacks essential elements of viral replication. Nevertheless, the VLPs according to the invention comprise genetic elements that are essential for VLP assembly. In a preferred embodiment, the VLPs comprise only those elements of the viral genome that are essential for the assembly of the VLP and no further elements of the viral genome. For example, if the rod-shaped virus is TMV, then the VLP comprises preferably only a TMV origin-of-assembly sequence and no further TMV sequences. More preferably, the rod-shaped virus is TMV, the VLP comprises only a TMV origin-of-assembly sequence and no further TMV sequences and the origin-of-assembly is a sequence having a length of up to 300 nucleotides comprising:
(A) the sequence of SEQ ID NO:2; or
(B) a sequence having at least 85% identity to the sequence of SEQ ID NO:2; or
(C) a fragment of (A) or (B), wherein the fragment has a length of at least 150 nucleotides.

The VLPs are rod-shaped, i.e. the VLP does not have a spherical, such as a globular or an icosahedral, structure. Thus, the rod-shaped VLPs have a roughly constant diameter, whereas the length of the virus depends on the length of the comprised ribonucleic acid molecule. The rods comprise e.g. coat proteins in a helical structure that includes the ribonucleic acid molecule. The location of the ribonucleic acid molecule can be inside the helix or on the outside. The diameter of the VLP is preferably about 18 nm, usually between about 10 and about 25 nm. For example, the VLP may have a length of at least 10 nm, at least 50 nm, at least 100 nm, at least 150 nm, at least 200 nm, at least 250 nm, at least 300 nm, at least 350 nm, at least 400 nm, at least 450 nm, or at least 500 nm. Preferably, the VLP has a length of at least 300 nm. More preferably, the VLP has a length of at least 500 nm. The length of the VLP may be e.g. 10-2000 nm (i.e. from 10 nm to 2000 nm), 50-2000 nm, 300-2000 nm, 400-2000 nm, or 500-2000 nm. However, the VLPs may also be longer, such as 10-10000 nm, 50-10000 nm, 300-10000 nm, 400-10000 nm or 500-10000 nm.

The VLP is mixed with the sample by any standard mixing procedure. The mixing in step (a) can comprise a step (a1) of adding the VLP to the sample and/or a step (a2) of intermixing the VLP and the sample. In a preferred embodiment, step (a) comprises a step (a1) of adding VLP to a sample, and a step (a2) of intermixing the resulting solution. For example, VLP may be added to the sample by pipetting, pouring, diffusion, and the like. The intermixing, i.e. the formation of a homogenous mixture of sample and VLP, may be e.g. by pipetting, by shaking, such as gentle or vigorous shaking, by rolling and the like. However, mixing may also be by diffusion, e.g. waiting until VLP and sample have mixed through entropy or by diffusion that is enhanced with ultrasound or the like.

In step (a) a specific amount of VLP is used. A specific amount is an amount if the quantity is known or can readily be determined before or at the time of mixing. The advantage of this way of proceeding resides in the fact that the heterologous RNA comprised in the VLP can be used as a standard. The VLP may for example be used in a known concentration and volume. The person skilled in the art is aware of methods for the determination of concentrations or absolute quantities of VLPs. The number of coat proteins and the number of ribonucleic acid molecules correlate with the number of VLP particles. The number of coat proteins correlates with the number of VLP molecules because the number of coat proteins in a single VLP is determined by the length of the enclosed ribonucleic acid molecule which is known. Therefore, a method suitable for the determination of the concentration or quantity of coat protein or of ribonucleic acid is also suited to determine the concentration or quantity of VLP and vice versa. Methods which allow determination of the concentration or quantity of coat protein are e.g. standard protein concentration measurements, such as measuring UV-absorption, detection with xanthoprotein, according to Millon, with ninhydrin, with biuret, the Bradford test, determination according to Lowry, the BCA reaction, and the like. Methods for the determination of concentration or quantity of ribonucleic acid are e.g. measuring optical density at OD₂₆₀ₙₘ and OD₂₈₀ₙₘ, measuring fluorescence, electrophoretic analysis, capillary electrophoresis, determination of the phosphate content, quantification of the nucleotide amounts after enzymatic hydrolysis of the RNA, quantitative RT-PCR analysis and the like. Optical densities at OD₂₆₀ₙₘ and OD₂₈₀ₙₘ can be derived from defined wavelength measurements or an UV spectrum. Fluorescence can be measured in combination with a nucleic acid binding dye like Ethidium bromide, SYBR and the like. Electrophoretic analysis can be carried out with a stained agarosegel. Capillary electrophoresis can be performed with instruments like the Agilent Bioanalyzer 2100, TapeStation 2200 and the like. Quantification of the nucleotide amounts after enzymatic hydrolysis of the RNA can be achieved by measuring optical density at OD₂₆₀ₙₘ and OD₂₈₀ₙₘ as described above. An instrument suitable for quantitative RT-PCR analysis is e.g. the RotorGene Q.

As indicated above, the VLPs comprise a ribonucleic acid molecule and a viral coat. In a preferred embodiment, the VLP consists of a ribonucleic acid and a viral coat. However, the VLP may further comprise other molecules, such as polyethylene glycol (PEG), peptides, and the like, that are attached by physical, ionic or covalent binding.

The ribonucleic acid (RNA) molecule is a chain of ribonucleotides. It is preferred that the RNA molecule consists of ribonucleotides. However, the RNA molecule may further comprise modified ribonucleotides (nt) which contain modifications of the ribose and/or the base. Suitable ribose modifications are e.g. selected from the group consisting of methylation, phosphate modifications, such as e.g. phosphothioates, and the like. Suitable base modifications are e.g. selected from the group consisting of methylation, such as methylation of the 5′ position of a Pyrimidine base, e.g. as in 5-Methylcytosine (m5C); isomerisation, e.g. as in pseudouridine (pseudo-U); and the like. Moreover, the RNA molecule may also comprise other chemical modifications, such as attachment of one of more biotin, PEG, peptides, inverted dinucleotides, such as a cap structure, and the like.

Preferably, the ribonucleic acid contained in the VLP is monopartite, i.e. the VLP contains a single molecule of ribonucleic acid. It is also preferred that the RNA molecule is single-stranded, i.e. ssRNA. However, as RNA molecules may have secondary and tertiary structural elements, this does not exclude that the RNA molecule can comprise sections forming e.g. double helices. For example the natural RNA genome of TMV (SEQ ID NO:6) forms a hairpin loop structure. It forms a helical structure around an RNA-free virus core. The structure of the RNA molecule comprised in the present VLP is, however, not limited by the above considerations, as long as the formation of the viral coat is not disturbed and protection against RNases and nucleases is maintained. The RNA molecule is linear, circular or branched, such as branched with one or more branches. In a preferred embodiment, the RNA molecule is linear.

Preferably, the RNA molecule is a ssRNA RNA molecule, such as a ssRNA(+) or a ssRNA(−) molecule.

The heterologous RNA sequence comprised by the RNA molecule may be a sense or an antisense sequence, i.e. have the desired sequence of bases or be the reverse complement of the desired sequence.

In principle, the RNA molecule is not subject to any size limitations because the length of the viral coat is adapted to the length of the RNA molecule. Therefore, the RNA molecule may be as short as about 150 or 200 ribonucleotides, which is approximately the length of the OAS. However, the RNA molecule may also have a length of several hundred or thousand ribonucleotides. For example, the RNA molecule can have a length of at least 150 ribonucleotides, at least 200 ribonucleotides, at least 350 ribonucleotides, at least 500 ribonucleotides, at least 1000 ribonucleotides, at least 2000 ribonucleotides, at least 5000 ribonucleotides, at least 6000 ribonucleotides, at least 7000 ribonucleotides, at least 8000 ribonucleotides or at least 9000 ribonucleotides. In a preferred embodiment, the RNA molecule has a length of at least 2000 ribonucleotides. It is further preferred that the RNA molecule has a length of at least 5500 ribonucleotides. The RNA molecule may even have a length of up to several thousand or several ten thousand ribonucleotides. Thus, the length of the RNA molecule may be e.g. 150-100,000 ribonucleotides, 200-100,000 ribonucleotides, 350-100,000 ribonucleotides, 500-100,000 ribonucleotides, 1000-100,000 ribonucleotides, 2000-100,000 ribonucleotides, 5000-100,000 ribonucleotides, 6000-100,000 ribonucleotides, 7000-100,000 ribonucleotides, 8000-100,000 ribonucleotides, or 9000-100,000 ribonucleotides.

The ribonucleic acid molecule comprises an origin-of-assembly sequence of a rod-shaped RNA virus and a heterologous sequence.

An OAS in the RNA molecule provides for the self-assembly of the VLP in vitro. Viral coat proteins initially bind to the OAS sequence in the RNA molecule and thereby provide an assembly initiation structure from which the VLP can subsequently grow by repeated addition of further coat proteins until the RNA molecule is completely encapsidated. The term “origin of assembly sequence” in the sense of the present invention does not necessarily refer to a full length wild type OAS sequence. Instead, it refers to any fragment or variant of a wild-type OAS sequence that ensures an efficient assembly and a good long term stability of the VLP. Methods for the assessment of an efficient assembly and a good long term stability are known in the art and e.g. also described elsewhere herein.

The OAS may be the OAS from any rod-shaped RNA virus, however, it is preferred that the OAS and the at least one type of coat protein are from the same virus. In a particularly preferred embodiment, the OAS and at least one of the at least one type of coat protein are from TMV, i.e. are a TMV OAS and at least one TMV coat protein. It will be understood that “TMV” refers to wild-type TMV unless otherwise mentioned. Thus, preferably the OAS and the at least one type of coat protein are from a wild-type TMV. In other words, the rod-shaped virus-like particle according to the invention and for use in the methods of the invention preferably comprises a ribonucleic acid molecule and a viral coat, wherein: (i) the ribonucleic acid molecule comprises an origin-of-assembly sequence of TMV and a heterologous sequence; and (ii) the viral coat comprises at least one type of coat protein of TMV.

Nevertheless, mutants or variants of the TMV OAS and mutants or variants TMV coat proteins can also be used in the method or the VLP of the invention. The mutants or variants of the TMV OAS and mutants or variants TMV coat proteins have the same or improved properties with regard to VLP assembly efficiency and/or VLP stability when compared with the wild-type OAS or wild-type coat protein. Thus, also alternative coat proteins or coat protein mixtures can be used.

In the most preferred embodiment of the invention, the VLP is derived from TMV and the OAS is a TMV OAS. The core of the TMV assembly sequence, i.e. the minimal OAS sequence required for assembly, was identified by Turner et al. (1988, J Mol Biol, 203:531-547) as the 75 nucleotide sequence (SEQ ID NO:9) endogenously located between residues 5444 and 5518 from the 5′end of the TMV genome (SEQ ID NO.:6). Thus, the OAS may comprise SEQ ID NO:9. In the context of the present invention, further fragments of the TMV OAS (SEQ ID NO:1, SEQ ID NO:2 and SEQ ID NO:10) were analyzed for assembly efficiency and stability of the resulting particles. SEQ ID NO:1 is endogenously located between residues 5420 and 5546 from the 5′end of the TMV genome (SEQ ID NO.:6). SEQ ID NO:2 is endogenously located between residues 5313 and 5546 from the 5′end of the TMV genome (SEQ ID NO.:6). SEQ ID NO:10 is endogenously located between residues 5100 and 5692 from the 5′end of the TMV genome (SEQ ID NO.:6). Thus, alternatively, the OAS may be e.g. SEQ ID NO.:2.

It has been surprisingly found in the context of the present invention that the length of the OAS is decisive for an efficient assembly of the rod-shaped virus-like particle, for the long-term stability of the particle and a universal applicability of the system to any heterologous RNA sequence. In particular, as outlined elsewhere herein, the inventors have surprisingly found that a 234 nt sequence (SEQ ID NO:2) of the OAS of TMV is particularly suitable for viral assembly and superior in the stability of the resulting particles and the assembly efficiently over OAS sequences with a length of 127 or 593 nucleotides (SEQ ID NO:1 and 10, respectively). While SEQ ID NO:2 has been found to be superior to SEQ ID NO:1 and SEQ ID NO:10, it will be understood that the TMV OAS according to the invention can also be slightly longer or shorter, i.e. a limited number of nucleotides can be added or deleted from the SEQ ID NO:2 without affecting the assembly efficiency or the long-term stability of the resulting VLPs.

Specifically, it has been found that the mentioned advantageous properties are achieved with OAS' having a length of from 150 to 300 ribonucleotides. Therefore, the OAS has a length of from 150 to 300, from 160 to 290, from 170 to 280, from 190 to 280, from 210 to 260, or from 230 to 240 ribonucleotides. In one embodiment, the OAS has a length of from 150 to 300 ribonucleotides. In a preferred embodiment, the OAS has a length of from 190 to 280 ribonucleotides. In a further preferred embodiment, the OAS has a length of from 210 to 260 ribonucleotides. For example, the rod-shaped virus-like particle according to the invention and for use in the methods of the invention preferably comprises a ribonucleic acid molecule and a viral coat, wherein: (i) the ribonucleic acid molecule comprises an origin-of-assembly sequence of TMV and a heterologous sequence; and (ii) the viral coat comprises at least one type of coat protein of TMV, wherein the origin-of-assembly is a sequence having a length of from 150 to 300 nts.

In other words, the OAS will have a length of up to 300 nucleotides. For example, the OAS has a length of up to 300, up to 290, up to 280, up to 270, up to 260, up to 250, or up to 240 ribonucleotides.

It is nevertheless preferred that the OAS comprises a sequence that has a length of at least 234 nucleotides. Therefore, the OAS preferably has a length of from 234 to 300, from 234 to 290, from 234 to 280, from 234 to 280, from 234 to 260, or from 234 to 240 ribonucleotides. In one embodiment, the OAS has a length of from 234 to 300 ribonucleotides. In a preferred embodiment, the OAS has a length of from 234 to 280 ribonucleotides. In a further preferred embodiment, the OAS has a length of from 234 to 260 ribonucleotides.

This means that the ribonucleic acid molecule comprises no further ribonucleotides or sequences that belong to the OAS of the rod shaped virus. The positions of the OAS in rod shaped viruses are well known or can be determined by standard testing methods that are known in the art. For example, it is known that the OAS of TMV can have a length of ˜440 bp (Sleat et al., 1986, Virology, 155:299-308). Methods that allow the discrimination of OAS sequences from nucleotides that belong to e.g. the heterologous sequence are well known in the art. For example, the sequence of an OAS may be obtained from a scientific article or a database. Alternatively, an OAS sequence can also be experimentally derived by e.g. sequencing experiments and functional analysis of deletion mutants. One example for the sequence of an OAS is SEQ ID NO:2. The OAS sequence can then be aligned to the ribonucleic acid molecule of the invention using a computer alignment tool and e.g. BLAST algorithm. Thereby the skilled person can easily determine whether a sequence in the ribonucleic acid molecule is an OAS, such as: (A) the sequence of SEQ ID NO:2; or (B) a sequence having at least 85% identity to the sequence of SEQ ID NO:2; or (C) a fragment of (A) or (B).

Preferably, the ribonucleic acid molecule comprises no further ribonucleotides (sequences) (other than the above defined OAS sequences) from the rod-shaped RNA virus. For example, if the rod-shaped virus is TMV, the ribonucleic acid molecule will comprise only nucleotides that belong to the TMV OAS and preferably no nucleotides from other regions of the TMV genome. The shortness of the OAS according to the invention provides the further advantage that the risk for interference of viral backbone sequences with downstream assays (e.g. RT-PCR assays that detect the heterologous sequences) is much reduced.

The OAS comprises preferably SEQ ID NO:2 or a homolog or a fragment thereof. In other words, the origin-of-assembly is a sequence having a length of up to 300 nucleotides comprising:

(A) the sequence of SEQ ID NO:2; or

(B) a sequence having at least 85% identity to the sequence of SEQ ID NO:2; or

(C) a fragment of (A) or (B), wherein the fragment has a length of at least 150 nucleotides.

For example, the rod-shaped virus-like particle according to the invention and for use in the methods of the invention preferably comprises a ribonucleic acid molecule and a viral coat, wherein: (i) the ribonucleic acid molecule comprises an origin-of-assembly sequence of TMV and a heterologous sequence; and (ii) the viral coat comprises at least one type of coat protein of TMV, wherein the origin-of-assembly is a sequence having a length of up to 300 nucleotides comprising:

(A) the sequence of SEQ ID NO:2; or

(B) a sequence having at least 85% identity to the sequence of SEQ ID NO:2; or

(C) a fragment of (A) or (B), wherein the fragment has a length of at least 150 nucleotides.

In a preferred embodiment (A), the OAS comprises or consists of SEQ ID NO:2, i.e. has the sequence shown in SEQ ID NO:2. In this embodiment, the OAS is a sequence having a length of from 234 to 300 nucleotides. In further embodiments (B), the OAS is a sequence having a length of up to 300 nucleotides and at least 85% identity to the sequence of SEQ ID NO:2. Specifically, the sequence of SEQ ID NO:2 can be modified by addition, deletion or mutation of a number of nucleotides without affecting the ability of the OAS to induce an efficient assembly. The person skilled in the art is able to design suitable mutations. For example, it is known in the art that the OAS comprises hairpin structures with double stranded regions. A mutation is, thus, preferably a compensatory mutation, meaning that both nucleotides which form a base pair in a double strand are replaced by other nucleotides which are also able to form a base pair. As indicated above, the OAS sequence having at least 85% identity to the sequence of SEQ ID NO:2 have the same or improved properties with regard to VLP assembly efficiency and/or VLP stability when compared with SEQ ID NO:2.

The OAS sequence having a length of up to 300 nucleotides and at least 85% identity to the sequence of SEQ ID NO:2, can, thus, have at least 85%, 86%, 87%, 88%, 89%, 90%, 91%, 92%, 93%, 94%, 95%, 96%, 97%, 98%, or 99% identity to SEQ ID NO:2. In one embodiment, the OAS sequence having at least 85% identity to the sequence of SEQ ID NO:2 has at least 90% identity to SEQ ID NO:2. In a further preferred embodiment, the OAS sequence having at least 85% identity to the sequence of SEQ ID NO:2 has at least 95% identity to SEQ ID NO:2. In a most preferred embodiment, the OAS sequence having at least 85% identity to the sequence of SEQ ID NO:2 has at least 99% identity to SEQ ID NO:2. In other words, the OAS sequence having at least 85% identity to the sequence of SEQ ID NO:2 has from 85% to 100%, 86% to 100%, 87% to 100%, 88% to 100%, 89% to 100%, 90% to 100%, 91% to 100%, 92% to 100%, 93% to 100%, 94% to 100%, 95% to 100%, 96% to 100%, 97% to 100%, 98% to 100%, or 99% to 100% identity to SEQ ID NO:2. In still further embodiments, the OAS is a sequence having a length of up to 300 nucleotides, comprising (C) a fragment of (A) or (B), wherein the fragment has a length of at least 150 nucleotides. Specifically, said fragment has a length of at least 150, 160, 170, 180, 190, 200, 210, 220 or 230 nucleotides. In a preferred embodiment, the fragment has a length of at least 200 nucleotides. In a further preferred embodiment, the fragment has a length of at least 220 nucleotides.

The fragment of (A) or (B), i.e. the fragment of SEQ ID NO:2 or a sequence having at least 85% identity to SEQ ID NO:2, is a functional OAS. This means that the fragment has the same or improved properties with regard to VLP assembly efficiency and/or VLP stability when compared with SEQ ID NO:2. Preferably, the fragment comprises SEQ ID NO:9 or functional mutants and variants thereof, i.e. the minimal 75 nt sequence that is required for assembly for TMV particles. Functional mutants and variants of this sequence comprise e.g. compensatory mutations, and have the same or improved properties with regard to the assembly efficiency and the stability of the resulting VLP when compared with SEQ ID NO:9. As outlined above under (B), the functional mutants and variants of SEQ ID NO:9 will have at least 85% identity, preferably 90% identity, more preferably 95% identity to SEQ ID NO:9.

Further to SEQ ID NO:9, the fragment of (A) or (B) can comprise additional nucleotides 5′ and 3′ of SEQ ID NO:9. For example, it has been found in the context of the present invention that it is advantageous to add approximately 30 nucleotides from TMV OAS to the 3′ end of the SEQ ID NO:9. Specifically, it is preferred that the fragment of (A) or (B) comprises SEQ ID NO:9 and about 28 further consecutive nucleotides from TMV OAS at the 3′ end of SEQ ID NO:9. “About” 28 nucleotides means e.g. that 26, 27, 28, 29, or 30 nucleotides can be added. However, this does not exclude that further TMV OAS nucleotides may be added to the 3′ end of the SEQ ID NO:9. As outlined above under (B), the fragments can also comprise a sequence having at least 85% homology to the SEQ ID NO:9 and the about 28 further consecutive nucleotides from TMV OAS at the 3′ end of SEQ ID NO:9. Preferably, the fragment of (A) or (B) comprises SEQ ID NO:9 and (not more than) 28 consecutive nucleotides from TMV OAS at the 3′ end of SEQ ID NO:9, or a sequence having at least 85% identity thereto.

Further to SEQ ID NO:9 and the above described 3′ sequences, or a sequence having at least 85% thereto, the fragment of (A) or (B) can comprise additional nucleotides from the TMV OAS 5′ of the SEQ ID NO:9. For example, the fragment can comprise from 47 to 197 consecutive nucleotides from the TMV OAS at the 5′ end of SEQ ID NO:9, or a sequence having at least 85% identity thereto. Preferably, the fragment comprises about 131 consecutive nucleotides from the TMV OAS at the 5′ end of SEQ ID NO:9, or a sequence having at least 85% identity thereto (as in SEQ ID NO:2). Nevertheless, as the assembly starts at the 3′ end of the OAS, it will be understood that more nucleotides may be added 5′ from SEQ ID NO:9.

It will be understood that the OAS has the same or improved properties compared with SEQ ID NO:2 with regard to the assembly efficiency, the stability of the resulting particles and the ability to initiate packaging of substantially any heterologous RNA sequence.

The viral assembly starts at the 3′-end of the OAS. Endogenously, the OAS sequence of TMV is located 1 Kb from the 3′-end of the viral genomic RNA. It was surprisingly found that omission of non-essential 3′-sequences from the endogenous RNA genome of rod-shaped viruses, i.e. placement of the OAS at the very 3′-end, improves the assembly of the VLP. The inventors have established a method to construct DNA templates which permit this direct 3′-terminal location, without any sequence constraints in the desired RNA construct. This method uses insert-specific PCR and RNA synthesis from the generated PCR product. Details of the procedure are described in the example section herein below. Thus, in a particularly preferred embodiment of the invention, the OAS is directly at the 3′end of the ribonucleic acid molecule. “Directly” in this context means that essentially no further nucleotides are between the OAS sequence and the 3′-end of the ribonucleic acid molecule. However, it is to be understood that the ribonucleic acid molecule may contain a few nucleotides, such as 1, 2, 3, 4, 5, 6, 7, 8, 9 or 10 nucleotides, 3′ from the OAS sequence. Preferably, the ribonucleic acid molecule contains less than 10 nucleotides which do not belong to the OAS (non-OAS nucleotides) 3′ from the OAS sequence. In a particularly preferred embodiment of the invention, the VLP comprises a TMV coat protein and a TMV OAS, wherein the OAS is directly at the 3′end of the ribonucleic acid molecule (without a single additional 3′-terminal nt between the OAS and the 3′-end). For example, the rod-shaped virus-like particle according to the invention and for use in the methods of the invention preferably comprises a ribonucleic acid molecule and a viral coat, wherein: (i) the ribonucleic acid molecule comprises an origin-of-assembly sequence of TMV and a heterologous sequence; and (ii) the viral coat comprises at least one type of coat protein of TMV, wherein the origin-of-assembly is a sequence having a length of up to 300 nucleotides comprising: (A) the sequence of SEQ ID NO:2; or (B) a sequence having at least 85% identity to the sequence of SEQ ID NO:2; or (C) a fragment of (A) or (B), wherein the fragment has a length of at least 150 nucleotides; and wherein the OAS is directly at the 3′-end of the ribonucleic acid molecule (without a single additional 3′-terminal nt). In one particularly preferred embodiment, the rod-shaped VLP according to the invention and for use in the methods of the invention preferably comprises a ribonucleic acid molecule and a viral coat, wherein: (i) the ribonucleic acid molecule comprises an origin-of-assembly sequence of TMV and a heterologous sequence; and (ii) the viral coat comprises at least one type of coat protein of TMV, wherein the origin-of-assembly is a sequence having a length of up to 300 nucleotides comprising: the sequence of SEQ ID NO:2, and wherein the OAS is directly at the 3′-end of the ribonucleic acid molecule (without a single additional 3′-terminal nt).

The length and the position of the OAS confer a particularly high stability to the resulting rod-shaped virus-like particles. Thus, the particles of the present invention are stable during storage. “Stable” means that at least 50%, preferably at least 70%, more preferably at least 90% of all virus-like particles remain intact during storage, i.e. are not degraded by RNases and the like. Methods for the detection of intact virus-like particles are known in the art and are described elsewhere herein. For example, the stability of the particles can be measured by real time RT-PCR. The particles are regarded as stable in case the cycle threshhold value Ct in real-time RT-PCR assays does not change by more than 1 unit during storage. Thus, in a preferred embodiment, the VLP of and for use in the methods of the invention is stable, wherein a VLP is regarded as stable in case the cycle threshhold value Ct in real-time RT-PCR assays does not change by more than 1 unit during storage.

Storage may e.g. be from −30° C. to −10° C., such as at about −20° C. Storage may include shipments on dry ice. Storage may also include several freeze-thaw cycles (as is required for a commercial product). Further, storage can be up to several months. For example, storage and/or handling can be at 37° C. for up to 3 weeks or at −20° C. for more than 12 months. Thus, for testing the stability of a VLP, storage of the VLP may e.g. be at from −30° C. to −10° C. for 20 to 25 days. Thus, in a preferred embodiment, the VLP of and for use in the methods of the invention is stable, wherein a VLP is regarded as stable in case the cycle threshold value Ct in real-time RT-PCR assays does not change by more than 1 unit during storage, wherein storage is at from −30° C. to −10° C. for 20 to 25 days. Nevertheless, it was shown that the VLPs are also particularly stable at higher temperatures. Thus, in a different embodiment, the VLP of and for use in the methods of the invention is stable, wherein a VLP is regarded as stable in case the cycle threshold value Ct in real-time RT-PCR assays does not change by more than 1 unit during storage, wherein storage is at about 37° C. for 20 to 25 days.

The rod-shaped RNA virus is preferably a plant-specific virus, preferably selected from the group consisting of viruses of the Virgaviridae family, such as viruses from the genera Furovirus, Hordeivirus, Pecluvirus, Pomovirus, Tobamovirus, and Tobravirus. It is particularly preferred that the rod-shaped virus is a virus of the genus Tobamovirus. Viruses of the genus Tobamovirus are e.g. Tobacco Mosaic Virus (TMV), Cucumber green mottle mosaic virus (CGMMV), Pepper mild mottle virus (PMMoV), Odontoglossum ringspot virus (ORSV) and Tomato mosaic virus (ToMV). In a particularly preferred embodiment, the rod-shaped RNA virus is TMV, such as e.g. the TMV represented by the genome sequence depicted in SEQ ID NO:6.

Further, the plant-specific virus may also be a virus of the Alphaflexiviridae family of viruses, such as a virus from the genera Allexivirus, Botrexvirus, Lolavirus, Mandarivirus, Potexvirus (e.g. Potato virus X (PVX), Clover yellow mosaic virus (ClYMV), Cymbidium mosaic virus (CymMV)), and Sclerodarnavirus. Alternatively, the plant-specific virus may also be a virus of the Potyviridae family of viruses, such as a virus from the genera Brambyvirus, Bymovirus, Ipomovirus, Macluravirus, Poacevirus, Potyvirus, Rymovirus, and Tritimovirus.

The heterologous sequence comprised by the RNA molecule may be any RNA sequence. “Heterologous” in the sense of the present invention is a sequence that is not from the genome of said rod-shaped RNA virus. That means that the RNA molecule is not an endogenously occurring viral sequence, but is generated experimentally.

Methods for the generation of RNA molecules comprising a viral OAS and a heterologous sequence are known to the person skilled in the art. Any standard cloning method may be used, such as those described by Sambrook et al. (2000, Molecular Cloning, A laboratory manual, 3^{rd} ed., Cold Spring Harbour Laboratory). For example, the RNA molecule may be generated by transcription, such as in vitro transcription, from a DNA template. The DNA template may be any DNA molecule, such as a circular or a linear DNA molecule, comprising a sequence encoding the RNA molecule. Preferably, the DNA template is double stranded.

Suitable transcription vector systems are known in the art. Suitable vectors comprise preferably an RNA polymerase promoter sequence, such as for example T7, SP6, and/or T3. The RNA polymerase promoter sequence may be comprised in the vector as distributed or inserted into the vector with a synthetic sequence comprising e.g. the RNA polymerase promoter sequence followed by the heterologous sequence and further followed by an OAS. Thus, for example, the pGEM vectors (from Promega) can be used.

For example, the heterologous sequence may be a marker sequence, a eukaryotic mRNA sequence, a synthetic sequence and/or a sequence from a pathogen. The pathogen is preferably selected from the group consisting of HIV-1, HIV-2, HCV, HTLV-1, HTLV-2, hepatitis G, an enterovirus, or a blood-borne pathogen.

As described above for the RNA molecule, also the heterologous sequence is in principle not limited in its size. The sequence may be as short as a few ribonucleotides, such as 1, 2, 3, 4, 5, 6, 7, 8, 9 or 10 ribonucleotides. However, the heterologous sequence can also be longer, such as at least 200, at least 500, at least 1000, at least 1100, at least 1200, at least 1300, at least 1400, at least 1500, at least 1600, at least 1700, at least 1800, at least 1900, at least 2000, at least 5000, at least 6000, at least 7000, at least 8000 or at least 9000 ribonucleotides. In a preferred embodiment, the heterologous sequence has a length of at least 1200 ribonucleotides. In a further preferred embodiment, the heterologous sequence has a length of at least 2000 ribonucleotides. The heterologous sequence may even have a length of up to several thousand or several ten thousand ribonucleotides. Thus, the length of the heterologous sequence may be e.g. 200-100,000 ribonucleotides, 350-100,000 ribonucleotides, 500-100,000 ribonucleotides, 1000-100,000 ribonucleotides, 2000-100,000 ribonucleotides, 5000-100,000 ribonucleotides, 6000-100,000 ribonucleotides, 7000-100,000 ribonucleotides, 8000-100,000 ribonucleotides, or 9000-100,000 ribonucleotides.

In consequence, a preferred embodiment of the invention provides a method for the detection of RNA in a sample, comprising
(a) mixing a specific amount of a TMV virus-like particle with a sample, wherein the TMV particle comprises a ribonucleic acid molecule and a viral coat, wherein:
   (i) the ribonucleic acid molecule comprises a TMV origin-of-assembly sequence and a heterologous sequence, wherein the heterologous sequence has a length of 2000-100,000 ribonucleotides and the origin-of-assembly is a sequence having a length of up to 300 nucleotides comprising:
      (A) the sequence of SEQ ID NO:2; or
      (B) a sequence having at least 85% identity to the sequence of SEQ ID NO:2; or
      (C) a fragment of (A) or (B), wherein the fragment has a length of at least 150 nucleotides; and
   (ii) the viral coat comprises at least one type of coat protein of TMV;
(b) isolating RNA from the sample; and
(c) detecting RNA comprising the heterologous sequence.

In a preferred embodiment of the method of the invention, the heterologous sequence is not present in the sample before mixing in step a). Then, there is no background signal that could hypothetically distort the detection of the heterologous sequence. However, it is evident that small amounts of heterologous sequence in the sample before mixing do not significantly affect the outcome of the present method.

The RNA molecule can also comprise more than the above sequences, i.e. more than the origin-of-assembly sequence of a rod-shaped RNA virus and a heterologous sequence. For example, the RNA molecule may comprise one or more additional sequences of the rod-shaped RNA virus. Further, the RNA molecule may comprise one or more additional heterologous sequences, for example selected from the group consisting of viral RNA sequence segments, e.g. derived from viruses like HIV, HCV, Influenza, and the like; mRNA sequence segments, such as segments of mRNAs encoding bcr/abl, EGFR, and the like; a synthetic control sequence; and others. Preferably, the synthetic control sequence is not present in the sample.

In one embodiment of the invention, the RNA molecule comprises at least one additional sequence, that assists, i.e. improves and/or accelerates, the assembly of the VLP. Sequences that assist in the assembly of the VLP can e.g. be selected from the group consisting of a 5′-cap structure; a combination of (i) a universal 5′-leader and (ii) a complementary sequence to the 5′-leader; and a poly(A) stretch.

In a preferred embodiment, the RNA molecule comprises a 5′-cap structure located at the 5′-end of the molecule. A 5′-cap structure in the sense of the present invention comprises a guanosine nucleoside connected to the RNA molecule via a 5′ to 5′ triphosphate linkage. Suitable 5′-cap structures are e.g. selected from the group consisting of m⁷Gppp (endogenous cap), m⁷GpppGm (endogenous cap 1 structure), 3′-O-methyl-m⁷Gppp (cap structure of an anti-reverse cap analogue (ARCA)), and 2′-O-methyl-m⁷Gppp (cap structure of an ARCA). “m” is methylation of the indicated position in the base of the nucleotide. “G” is guanosine. “p” is a phosphate group. “O” is oxygen.

In a further embodiment, the RNA molecule comprises a universal 5′-leader located at or near the 5′-end of the RNA molecule and a complementary sequence to the 5′-leader. The complementary sequence to the 5′-leader can be located between the heterologous sequence and the OAS or at or near the 3′-end of the RNA molecule. “Near” is to be understood as location in a distance of less than 500 nucleotides, preferably less than 100 nucleotides and more preferably less than 50 nucleotides. The universal 5′-leader may be e.g. that depicted in SEQ ID NO:3. The complement to the 5′leader will then be the sequence depicted in SEQ ID NO:4 or a variant thereof. Preferably, the variant comprises one or more segments of SEQ ID NO:4, with a minimum uninterrupted length of 6 ribonucleotides per segment.

In yet another embodiment, the RNA molecule comprises a poly(A)-stretch, e.g. adjacent to the OAS sequence. “Adjacent” means either 5′ of or 3′ of. Further, “adjacent” means that no or only a limited number of nucleotides, such as e.g. less than 10, less than 20, less than 30, less than 40, less than 50, less than 60, less than 70, less than 80, less than 90 or less than 100, are between the sequences.

The viral coat is the protein shell of a virus or of a VLP. Usually, it consists of several protein monomers, the coat proteins, of the same coat protein type or different types of coat proteins. The viral coat comprises at least one type of coat protein of the rod-shaped RNA virus. Thus, the viral coat may comprise 1, 2, 3, 4 or more types of coat proteins of the rod-shaped RNA virus, such as between 1 to 5 types of coat proteins. Further, it may also comprise 2 or more types of coat proteins from different rod-shaped RNA viruses. A type of protein differs from another type of coat protein by its amino acid sequence.

Several types of coat proteins of rod-shaped viruses are described in the art. In a particularly preferred embodiment, the type of coat protein is the coat protein of TMV shown in SEQ ID NO.:5). This coat protein is encoded by the open reading frame in the TMV genome (SEQ ID NO:6) at positions 5712-6191.

The present invention therefore provides VLPs comprising or consisting of TMV coat proteins comprising SEQ ID NO:5 and ssRNA comprising a heterologous sequence and a TMV OAS comprising SEQ ID NO:2. The invention further provides the use of these VLPs in the methods and uses as described above.

In another embodiment The present invention therefore provides VLPs comprising or consisting of TMV coat proteins comprising SEQ ID NO:5 and ssRNA comprising a 5′cap-structure, a heterologous sequence, a universal 5′leader and a TMV OAS comprising SEQ ID NO:2 and a poly(A) stretch. The invention further provides the use of these VLPs in the methods and uses as described above.

In a further embodiment the type of coat protein may be a wild-type coat protein or a genetically and/or chemically modified coat protein. Modified coat proteins may e.g. be mutants or variants of wild-type coat proteins. For example, amino acid changes in the sequence of the coat protein may be made on the basis of similarity in polarity, charge, solubility, hydrophobicity, and/or amphiphatic nature with the original amino acid. Alternatively, the amino acid change introduces a reactive moiety, such as the amino group in lysine, the thiol group in cysteine or the hydroxyl group in serine. Furthermore, the modification may be the introduction of a marker molecule into the coat protein, such as e.g. biotin, PEG, and the like.

The term “mutant” should be understood as a functionally equivalent protein, e.g. a mutant that has substantially the same, or improved, properties regarding the assembly of the protein into a VLP and the protection of the RNA in the assembled VLP compared with the endogenous coat protein. “Improved” in the present context means usually a faster and/or more stable assembly into the VLP and/or a better protection of the RNA. However, it may also be an improvement to generate a VLP that is less stable, for example to ease the intracellular release of the RNA molecule. All these mutants are a part of the present invention. In a presently preferred mutant, less that 5%, or less than 1% or even less than 0.1% of the amino acids in the protein have been shifted with another amino acid, or deleted, compared to the endogenous coat protein.

In the present context, the term “variant” should be understood as a protein which is functionally equivalent to a coat protein of the invention, e.g. having substantially the same, or improved, properties e.g. regarding the assembly of the protein into a VLP and the protection of the RNA in the assembled VLP. Such variants, which may be identified using appropriate screening techniques, are a part of the present invention.

The absolute number of coat protein monomers in the VLP according to the invention depends on the length of the RNA molecule. For example, the naturally occurring TMV has 2130 monomers of coat protein in its capsid shell and a genome length of approximately 6400 ribonucleotides. There are 3 ribonucleotides per coat protein monomer in the TMV. Thus, in a preferred embodiment, the number of coat proteins is about 1 per 3 ribonucleotides in the RNA molecule.

Preferably, the viral coat is a rod-like helical structure around the RNA molecule. For example, the rod-like helical structure may be built up from protein monomers. The protein monomers are coat proteins.

Methods for the manufacture of viral coat proteins are known in the art. For example, the proteins may be isolated from virus preparation made from infected plants, expressed in vitro in cell free systems or in eukaryotic or bacterial cells, such as E. coli or yeast, for example as described by Kadri et al. (2013, J Virol Methods, 189: 328-340). Corresponding expression constructs can be cloned by standard methods of the art.

In step b) of the method of the invention, RNA is isolated from the sample. Suitable methods for RNA isolation are known in the art. Any standard RNA isolation method known in the art is suitable to liberate RNA from the VLP as well as to isolate the RNA from the sample. For example, RNA may be isolated according to the acid guanidinium thiocyanatephenol-chloroform extraction method developed by Chomczynski and Sacchi, which is implemented e.g. in the Trizol kit (Life Technologies); with the Nonidet P-40 method; with column-based RNA isolation kits that are commercially available, and the like. In a preferred embodiment of the invention, the RNA is isolated in step b) with a column-based RNA isolation kit. Suitable column based kits for RNA isolation are e.g. RNeasy kits and QIAamp viral RNA kits (Qiagen).

It is preferred that the RNA is substantially pure after isolation, i.e. it essentially does not contain any compounds that would interfere with subsequent RNA sample processing, like nucleases, denaturing reagents like GTC or phenol, organic solvents like alcohols or chloroform. Furthermore, it is preferred that the preparation of isolated RNA is substantially free of desoxyribonucleic acid (DNA) and small RNAs, such as tRNAs and 5S rRNA.

Naturally, the skilled person will take care during isolation and subsequent handling of the RNA to avoid a contamination with ribonucleases (RNases). Means to avoid contamination are also well known in the art and include e.g. the use of sterile, RNase-free material, RNA stabilizing agents and frozen storage of the RNA.

Further, step c) is preferably carried out immediately after step b). Alternatively, the isolated RNA can be stored frozen as aqueous solution or as suspension in ethanol precipitation until detection of the RNA.

In step c) of the method according to the invention, the RNA comprising the heterologous sequence is detected. Several methods for the detection of heterologous sequences are known in the art. Any method for the detection of specific RNA sequences is suitable. Before carrying out one of the below detection methods, the RNA may be converted into DNA by reverse transcription. Ingredients and protocols for reverse transcription reactions are widely known in the art.

For example, the heterologous RNA sequence can be detected with sequence-specific primers or probes. Sequence-specific primers can be used e.g. in polymerase chain reaction (PCR) or other nucleic acid amplification methods like nucleic acid sequence based amplification (NASBA). Sequence-specific probes may be used e.g. in Northern blot, or modern versions with very high sensitivities such as branched DNA, e.g. in QuantiGene Assays (Affymetrix/Panomics) or NCounter technology (NanoSphere).

In a particularly preferred embodiment, the detection of RNA is a quantitative detection, i.e. the method comprises the quantitative detection of RNA. Methods for the quantitative detection of RNA are also widely known in the art. Quantitative detection can be by achieved with the same methods, i.e. by quantitative real-time RT-PCR, quantitative NASBA assays or quantitative probe-based assays, such as assays based on branched DNA, like e.g. QuantiGene Assays (Affymetrix/Panomics) or NCounter technology (NanoSphere).

In a further embodiment, the method for the detection of RNA in a sample further comprises the detection of a second RNA of interest in the sample. This RNA of interest is preferably an RNA from the sample. For example, the RNA of interest may be a marker RNA, such as a marker for a viral infection, a housekeeping gene transcript, or a biomarker for tumor-properties.

The detection of this RNA of interest allows the use of the method to standardize the quantity of the RNA of interest. The RNA molecule comprised in the VLP is protected against RNases. Consequently, the specific amount of VLP and, thus, heterologous RNA sequence will remain constant during handling of the mixture of sample and VLP until isolation of the RNA from the VLP. The heterologous RNA sequence can, thus, be used as an internal RNA standard or as a positive control. The detected amount of the second RNA of interest can be normalized against the detected amount of heterologous RNA sequence. Thus, in a still further embodiment, the method comprises a step, wherein the ratio between RNA of interest and the RNA with the heterologous sequence is determined.

In a further embodiment, the method comprises another step wherein the ratio between RNA of interest and the RNA with the heterologous sequence is compared with a reference ratio that was determined using the method of the invention at a different time and/or with a different sample. For example, one sample may be divided into two or more parts to which the method of the invention is applied at different times. This way, for example, a contamination with RNases may be tested. It is further possible to compare a ratio and a reference ratio that were determined with samples that were obtained from the same patient at different time points to monitor e.g. disease development. Moreover, ratio and reference ratio may also be determined from samples of a healthy and an individual having a malignancy.

To avoid any potential inaccuracies due to methodical mistakes, it is further preferred that both RNA detections are carried out by the same detection methods. Thus, it is preferred that the RNA of interest and the RNA with the heterologous sequence are detected by the same detection method. Specifically, the amounts of both RNAs are determined by the same detection methods. The detection method can therefore be any method described above being suitable for the detection of heterologous RNA sequences.

According to this aspect, the present invention thus provides methods for the quantitative detection of RNA in a sample, comprising
(a) mixing a specific amount of a TMV virus-like particle with a sample, wherein the TMV particle comprises a ribonucleic acid molecule and a viral coat, wherein:
   (i) the ribonucleic acid molecule comprises a TMV origin-of-assembly sequence and a heterologous sequence, wherein the heterologous sequence has a length of 2000-100,000 ribonucleotides and the origin-of-assembly is a sequence having a length of up to 300 nucleotides comprising:
      (A) the sequence of SEQ ID NO:2; or
      (B) a sequence having at least 85% identity to the sequence of SEQ ID NO:2; or
      (C) a fragment of (A) or (B), wherein the fragment has a length of at least 150 nucleotides; and
   (ii) the viral coat comprises at least one type of coat protein of TMV;
(b) isolating RNA from the sample; and
(c) detecting RNA comprising the heterologous sequence and a second RNA of interest in the sample, wherein the ratio between RNA of interest and the RNA with the heterologous sequence is determined by the same detection method.

The detected RNA amounts may be absolute or relative values. As described above, it is preferred that the detection is quantitative. Hence, the detection of RNA results preferably in absolute values. Such absolute values can be expressed e.g. in terms of weight, such as in gram, in terms of molecule number, such as in molar amounts, or in copy numbers. Nevertheless, the detection of RNA can also result in relative values. For example, during quantitative real time PCR the detection with different primer sets does not lead to absolute values of the template concentration. Therefore, the values can be normalized to a standard value, such as detected house-keeping gene(s). The same purpose may be served by the VLP encoated RNA molecules.

In another aspect, the present invention relates to the use of a rod-shaped virus-like particle as an internal RNA standard or as a positive control for RNA degradation and/or quantification, wherein the rod-shaped virus-like particle comprises a ribonucleic acid molecule and a viral coat, wherein: (a) the ribonucleic acid molecule comprises an origin-of-assembly sequence of a rod-shaped RNA virus and a heterologous sequence; and (b) the viral coat comprises at least one type of coat protein of the rod-shaped RNA virus. As outlined before, the origin-of-assembly sequence preferably has a length of from 150 to 300 nucleotides. More preferably, the origin-of-assembly is a sequence having a length of up to 300 nucleotides comprising: (A) the sequence of SEQ ID NO:2; or (B) a sequence having at least 85% identity to the sequence of SEQ ID NO:2; or (C) a fragment of (A) or (B), wherein the fragment has a length of at least 150 nucleotides. Moreover, the origin-of-assembly sequence is preferably directly at the 3′end of the ribonucleic acid molecule.

In an experiment, a positive control serves as a verification of the methodology used. In the method of the invention, the RNA molecule encapsidated in the VLP is protected from RNases and will lead to a detection of heterologous sequence. Thus, the person carrying out the method can be sure that a lack of detection of the heterologous sequence would be due to other mistakes made during the procedure. Consequently, the VLPs of the present invention are highly suited to serve as positive controls for RNA degradation and/or quantification.

In a different aspect, the present invention relates to the use of a rod-shaped virus-like particle for expression of the heterologous sequence in vitro, such as in a cell free system, or in host cells. The host cell is preferably selected from the group consisting of plant cells, animal cells, and yeast cells. In some embodiments, this use comprises the infection or transfection of the host cell with the VLP.

In a further aspect, the present invention relates to a rod-shaped virus-like particle comprising a ribonucleic acid molecule and a viral coat, wherein: (a) the ribonucleic acid molecule comprises an origin-of-assembly sequence of a rod-shaped RNA virus and a heterologous sequence; and (b) the viral coat comprises at least one type of coat protein of the rod-shaped RNA virus, wherein the origin-of-assembly sequence has a length of from 150 to 300 nucleotides. More preferably, the origin-of-assembly is a sequence having a length of up to 300 nucleotides comprising: (A) the sequence of SEQ ID NO:2; or (B) a sequence having at least 85% identity to the sequence of SEQ ID NO:2; or (C) a fragment of (A) or (B), wherein the fragment has a length of at least 150 nucleotides. In a preferred embodiment, the origin-of-assembly sequence comprises or consists of SEQ ID NO:2 or a sequence having at least 85% sequence identity thereto. In a particularly preferred embodiment, the present invention relates to a rod-shaped virus-like particle comprising a ribonucleic acid molecule and a viral coat, wherein: (a) the ribonucleic acid molecule comprises an origin-of-assembly sequence of a rod-shaped RNA virus and a heterologous sequence; and (b) the viral coat comprises at least one type of coat protein of the rod-shaped RNA virus; wherein the rod-shaped RNA virus is TMV; wherein the origin-of-assembly is a sequence having a length of up to 300 nucleotides comprising: (A) the sequence of SEQ ID NO:2; or (B) a sequence having at least 85% identity to the sequence of SEQ ID NO:2; or (C) a fragment of (A) or (B), wherein the fragment has a length of at least 150 nucleotides; and wherein the origin-of-assembly sequence is directly at the 3′ end of the ribonucleic acid molecule.

It is known in the art that rod-shaped viruses, such as TMV, can be assembled in vitro from an RNA molecule comprising an OAS and coat protein in a spontaneous reaction. This assembly is initiated at the OAS. In principle, the assembly process is independent from and not substantially affected by the sequence or the length of the remaining RNA molecule. Nevertheless, in particular the assembly with long RNA molecules or RNA having strong secondary and tertiary structures can be improved by the below manufacturing methods.

Generally beneficial conditions for the in vitro assembly of rod-shaped viruses are described in the art, e.g. by Butler (1984, J Gen Virol, 65:253-279), by Durham (1972, J Mol Biol, 67:289-305), by Wu et al. (2010, ACS Nano, 4:4531-8) and by Mueller et al. (2010, J Virol Methods, 166: 77-85) and can be used in the manufacturing methods of the present invention. For example, in one embodiment of the below manufacturing methods the pH is about 7. The below manufacturing methods are in vitro methods. The below manufacturing methods do not use a bacterial or eukaryotic expression system during the mixing step.

Thus, in yet another aspect the present invention is concerned with a method for the manufacture of a rod-shaped virus-like particle comprising a ribonucleic acid molecule and a viral coat, comprising a) mixing a ribonucleic acid molecule and at least one type of coat protein of a rod-shaped virus, wherein the ribonucleic acid molecule comprises an origin-of-assembly sequence of a rod-shaped RNA virus and at least one heterologous sequence; b) heating the ribonucleic acid molecule and/or the at least one type of coat protein before step a) and/or heating the mixture at least once after step a) to a temperature between about 30° C. and about 100° C., preferably to about 70° C.

In step a) of this manufacturing process a ribonucleic acid molecule and at least one type of coat protein of a rod-shaped virus are mixed. The ribonucleic acid molecule and the at least one type of coat protein have the properties described above for the method for the detection of RNA in a sample. The mixing may be achieved by methods known in the art. Methods suitable for mixing are for example those described above for the mixing of VLP and sample.

In step b) of this manufacturing process the ribonucleic acid molecule and/or the at least one type of coat protein is heated before step a) and/or the mixture is heated at least once after step a) to a temperature between about 30° C. and about 100° C., preferably to about 70° C. The inventors surprisingly found that these heating steps are able to assist in the manufacture of VLP. Presumably, the heat breaks up certain strong secondary and/or tertiary structures of the protein and RNA and thereby assists in the assembly of the viral particle. Therefore, this manufacturing process is e.g. for RNA molecules with strong secondary and/or tertiary structures.

Heating may be achieved by standard means of the laboratory practice. For example, heating may be heating in a water bath, in a heating block, in an incubator, in a thermocycler, and the like.

Heating is done to a temperature that is suitable to break up the above described structures in a protein or RNA preparation. For example, heating may be to a temperature of between about 30° C. and about 100° C., between about 50° C. and about 90° C., or between about 60° C. and about 80° C. Preferably, heating of the ribonucleic acid molecule is to a temperature of about 70° C. Heating of the at least one coat protein or the mixture of RNA molecule and the at least one coat protein is preferably to about 40° C., more preferably to about 30° C.

In a preferred embodiment, the ribonucleic acid molecule is heated before step a) to a temperature between about 30° C. and about 100° C., preferably to about 70° C. In a further embodiment the at least one type of coat protein is heated before step a) to a temperature between about 30° C. and about 100° C., preferably to about 40° C., more preferably to about 30° C. In yet another embodiment, the mixture of RNA molecule and the at least one coat protein is heated at least once after step a) to a temperature between about 30° C. and about 100° C., preferably to about 40° C., more preferably to about 30° C.

Heating the mixture at least once means that the mixture may be heated 1 times, 2 times, 3 times, 4 times, 5 times, 6 times, 7 times, 8 times, 9 times, or more often. Thus, the mixture may be heated e.g. between 1 and 30, between 1 and 20, or between 1 and 10 times. It is particularly preferred to assist the VLP assembly by continuous heating and cooling during the assembly process.

Between the heating steps the mixture may cool off on its own or be cooled by external means. For example, the mixture may be cooled to 50° C., to 37° C., to room temperature, or to 4° C. between the heating steps.

Cooling may be by standard means of the laboratory practice. For example, cooling may be cooling in a water bath, in a heating block, in an incubator, in a thermocycler, on ice, in a freezer, in a fridge, in a cold room, and the like.

Heating may be e.g. for 30 sec, 1 min, 5 min, 10 min or longer. Cooling may be e.g. for 30 sec, 1 min, 5 min, 10 min, or longer.

Preferably, the RNA molecule, the at least one type of coat protein and/or the resulting mixture are in solution, such as in an aqueous solution. The person skilled in the art is aware of aqueous solutions that are suited for nucleic acid and protein molecules. For example, it is known that buffering of the solution to a certain pH keeps these biological molecules stable. Therefore, the solution may comprise a buffer e.g. selected from the group consisting of a phosphate buffer, a Tris (tris(hydroxymethyl)aminomethane) buffer, a HEPES (4-(2-hydroxyethyl)-1-piperazineethanesulfonic acid) buffer, etc. Further, the solution may contain a salt, such as a sodium or potassium salt.

In a further aspect, the invention provides a method for the manufacture of a rod-shaped virus-like particle comprising a ribonucleic acid molecule and a viral coat, comprising a) mixing a ribonucleic acid molecule and at least one type of coat protein of a rod-shaped virus, wherein the ribonucleic acid molecule comprises an origin-of-assembly sequence of a rod-shaped RNA virus and at least one heterologous sequence; and b) adding a denaturing compound and/or a detergent to the ribonucleic acid molecule and/or the at least one type of coat protein before step a) and/or to the mixture after step a).

It was surprisingly found by the present inventors that a detergent or denaturing compound assists in the assembly of VLP during the manufacture process.

The addition in step b) may be performed by standard methods in the art. For example, any addition method described above for the addition of VLP to the sample in step a1) of the method for detection of RNA in a sample can also be used for addition of the denaturing compound and/or a detergent in the present method for manufacture of a VLP. Presumably, the denaturing compound or detergent breaks up certain strong secondary and/or tertiary structures of the protein and RNA and thereby assists in the assembly of the viral particle. Therefore, this manufacturing process is e.g. for RNA molecules with strong secondary and/or tertiary structures

Suitable denaturing compounds are e.g. dimethylsulfoxid (DMSO), formamide, urea, guanidinium-isothiocyanate and alcohols, such as ethanol, isopropanol and glycerol. The skilled person will, however, be able to find further suitable denaturing compounds in a straightforward manner.

Depending on their denaturing strength, the denaturing compounds are added to a final concentration of between 0.1 to 50%, between 1 to 40%, between 5 to 30%, between 10 to 20%. It is evident that the stronger a denaturing compound is, the less of the aforementioned denaturing compounds will be used. For example, DMSO and alcohols, such as ethanol, isopropanol and glycerol, are known to be mild denaturing agents and will, thus, be added to a final concentration of between 0.1 to 50%. Urea, formamide and Guanidinium-isothiocyanate are known to be harsher denaturing compounds and will, thus, be added to a final concentration of between 0.1 to 10%.

Suitable detergents are e.g. selected from the group consisting of non-ionic surfactants, such as Triton X 100, and polysorbate surfactants, such as polysorbate 20 (Tween 20) or polysorbate 80 (Tween 80). The skilled person will, however, be able to find further suitable detergents in a straightforward manner.

The detergents are added to a final concentration of between 0.1 to 10%.

In a preferred embodiment, a denaturing compound and/or a detergent is added to the ribonucleic acid molecule before step a). In a further embodiment a denaturing compound and/or a detergent is added to the at least one type of coat protein before step a). In yet another embodiment, a denaturing compound and/or a detergent is added to the mixture of RNA molecule and at least one coat protein after step a).

The methods for the manufacture of a rod-shaped virus-like particle disclosed herein may further comprise a step, wherein the average length of the rod-shaped virus-like particle is determined at least once during or after assembly. As described above, the length of the rod-shaped VLP correlates directly with the length of the enclosed RNA molecule. The expected length of a VLP can be calculated on the basis of the number of ribonucleotides of the RNA molecules by dividing the number of ribonucleotides by 21. For example, a particle containing the complete TMV RNA (approximately 6400 nt) has a length of approximately 300 nm (Wu et al., 2010, ACS Nano, 4:4531-8). In consequence of the above described correlation between the virus length and the RNA molecule, the progression of the VLP assembly can be monitored by measurement of the length of the VLP. Methods that are suited for the determination of the average length of the VLP are e.g. electron microscopy, dynamic light scattering, turbidimetry and the like. Any further suitable technique can evidently also be used.

All literature cited herein is fully enclosed herein by reference.

This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### DESCRIPTION OF THE FIGURES

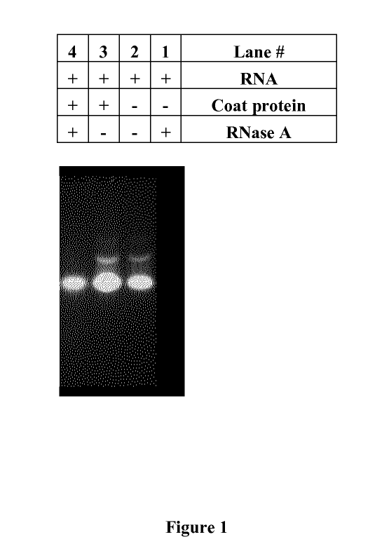

FIG. 1:
Agarose gel electrophoresis after following treatment:
Lane 1: Incubation of IVT-RNA (ca. 0.1 μg/μl) with RNase A (0.1 ng/μl) in the absence of coat protein, resulting in complete digestion. Lane 2: Control reaction in the absence of coat protein and in the absence of RNase. Lane 3: IVT-RNA (ca. 0.1 μg/μl) after assembly reaction with coat protein (ca. 2 μg/μl), without RNase treatment. Lane 4: IVT-RNA (ca. 0.1 μg/μl) after assembly reaction with coat protein (ca. 2 μg/μl) and with RNase treatment (ca. 0.1 ng/μl), degradation protection is evident.

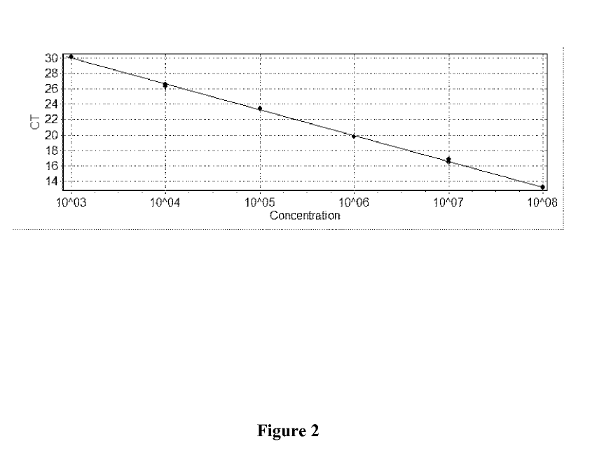

FIG. 2:
Standard curve with cDNA dilution series

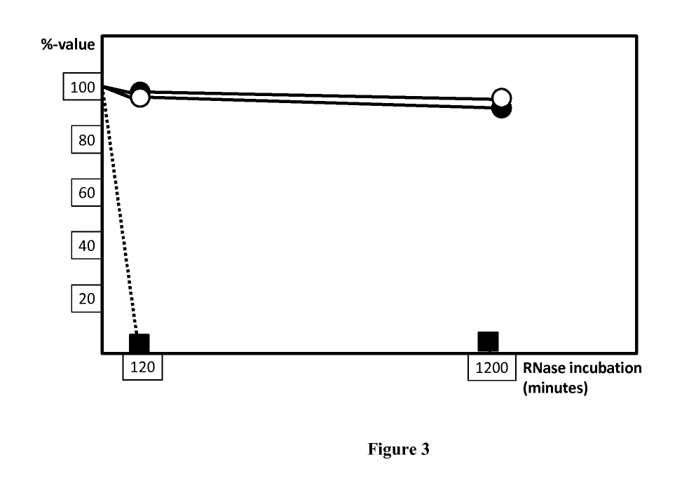

FIG. 3:
Incubation of Incoated RNA with RNases for prolonged periods. Unprotected, free RNA (▪, dotted line) was rapidly reduced to very low levels. Whereas no significant degradation and no significant differences with different RNase levels (1 U/assay, [symbol] 0.1 U/assay) were observed for RNA within Incoated RNA particles.

### EXAMPLES

The following examples are intended to illustrate the invention but not to limit its scope. Examples I to III provide procedures beginning with gene synthesis and leading to Incoated RNA particles, including quality control with RNase protection assays.

### Example I

### Synthesis of IVT-RNA for Assembly in “Incoated RNA”

The following experimental protocol has been carried out using numerous different sequences and is therefore illustrated in general terms of a general protocol:

A target sequence of interest was combined with two flanking sequence regions to obtain a “gene construct” with the following features:
5′-region: T7 promotor sequence (17 bp), followed by the universal 5′-leader sequence (78 bp)
core region: target sequence of interest (essentially any length is possible, hundreds or thousands of bp)
3′-region: OAS sequence of choice (234 bp)

### Schematic Presentation of the “Gene Construct”:

T7-promotor-5′-leader sequence-target of interest-OAS

The gene construct was obtained from a service provider (Eurofins MWG GmbH) which provides the construct as clone in a standard plasmid, for example pexA (Eurofins MWG GmbH). The clone of choice was fully characterised by complete sequencing of the insert and complete identity with the gene construct sequence ordered has been confirmed.

In the next step a template was generated by insert-specific PCR with a plasmid-derived primer, for example with the:
pexA-forward primer 5′-GGAGCAGACAAGCCCGTCAGG, and the
OAS-specific primer TMV-1 5′-CCGGTTCGAGATCGAAACTTTGC which hybridizes at the 3′-end of the OAS sequence element.

The resulting PCR product was purified and analysed by capillary electrophoresis.

The purified DNA template was used for IVT-reaction, and the resulting IVT-RNA product was purified and analysed by capillary electrophoresis.

a) Template DNA by Insert-Specific PCR
Plasmid-DNA (ca. 200 pg or ca. 0.1 fmole) was combined with 25 μl 2× Immomix (Bioline GmbH)+500 nM Primermix (equimolar pexA-forward+TMV-1) in a total volume of 50 μl. PCR was performed by initial incubation for 10 min at 95° C. for enzyme activation, followed by 30 cycles: 30 sec at 95° C., 25 sec at 51° C., 45 sec at 72° C., final extension with 4 min at 72° C.
The PCR products were purified by standard spin column procedures, using “High Pure PCR Product Purification Kit” (Roche Applied Science) or “DNA Clean & Concentrator Kit” (Zymo Research). Purified PCR products were analysed with Biophotometer (Eppendorf GmbH) and by capillary electrophoresis (Agilent Bioanalyzer 2100 or Tapestation 2200).

### b) IVT-RNA Synthesis

Purified PCR product (10 μl, ca. 10% of the generated PCR product from step (a), above) was used for IVT-reaction with MegaScript kit (Ambion) in a total volume of 25 μl WT reaction was performed overnight (ca. 16 h) at 37° C. Template DNA was digested by two subsequent incubations, each with 1 μl DNase 1 (Ambion) or 1 μl Turbo DNase (Ambion) and incubation for 30 min at 37° C.
The WT-RNA products were purified by standard spin column procedures, using “RNeasy Mini Kit” (Qiagen). Purified IVT-RNA products are analysed with Biophotometer (Eppendorf GmbH) and by capillary electrophoresis (Agilent Bioanalyzer 2100 or Tapestation 2200).

### Example IIA

### Assembly of IVT-RNA and TMV Coat Protein to Generate “Incoated RNA” Particles

TMV coat protein was obtained from infected tobacco plants, according to published procedures, see Mueller et al. (2010, J Virol Methods 166: 77-85).

For assembly of “Incoated RNA” particles, excess TMV coat protein in sodium potassium phosphate buffer (SPP, 50 mM, pH 7.2) was mixed directly with purified IVT-RNA in a weight ratio in the range of about 25:1 to 20:1 (protein:RNA) and incubated overnight (16-20 h) at 30° C.

In an alternative approach, the IVT-RNA was preheated for 10 min at 70° C., followed by a slow cooling process at a rate of 0.01° C./sec to 30° C. Subsequently the RNA was mixed with coat protein, as above. An aliquot of this assembly reaction was used to test for successful generation of “Incoated RNA” (following the methods described in example IIIA, IIIB, below).

“Incoated RNA” was recovered from the assembly mix by precipitation with PEG (polyethylene glycol) as follows:

PEG 6000 (final concentration of 4%) was added and incubated for at least 15 min on ice, followed by centrifugation for at least 15 min at 4° C. with 10,000×g to 20,000×g. Supernatant was discarded and the pellet was dissolved in 10 mM SPP buffer.

### Example IIB

### Assembly of IVT-16S rRNA and TMV Coat Protein to Generate “In-Coated RNA” Particles Comprising 16S rRNA

Two of many successfully used target sequences of interest were the 16S rRNA Domain I and the complete 16S rRNA sequence. Ribosomal RNA, rRNA is the RNA component of the ribosome and known to have particularly strong secondary structures which are presumably required for its function in the ribosome. Due to these strong secondary structures and the length of the RNA, it was thought that these RNAs could not be packaged into a viral particle.

The DNA templates used for insert-specific PCR as described in Example I above are shown in SEQ ID NO.:7 (16S rRNA Domain I) and in SEQ ID NO.:8 (complete 16S rRNA). In accordance with the above described procedure, the templates also comprise the 234 nt long OAS sequence (SEQ ID NO.:2) at the 3′ end. It was found that this OAS ensures an efficient assembly of the TMV-like particles and, further, that the resulting particles are also particularly stable (compare Example X below). All steps were carried out as described above.

Purified IVT-RNA products comprising either the 16S rRNA Domain I or the complete 16S rRNA sequence were analysed inter alia using an Agilent RNA Nano Kit, i.e. capillary electrophoresis, on the Agilent Bioanalyzer 2100. It was found that both RNAs were visible as single, clean peaks in the electropherogramm, demonstrating their integrity before the packaging procedure.

For assembly of “Incoated RNA” particles, excess TMV coat protein was mixed directly with the purified IVT-RNA as described above and incubated overnight (16-20 h) at 30° C. Using the RNase test described below in Example IIIB, it was shown that virus-like TMV particles were obtained during assembly that contained either the full length 16S rRNA Domain I or the full length 16S rRNA sequence. Specifically, the RNA constructs comprise the OAS at the 3′-end, followed by the rRNA sequence and the RT-PCR amplicon sequence is located after the rRNA sequence at the extreme 5′-end. Packaging begins at the 3′-end and protection of the amplicon sequence is only achieved after complete packaging of the RNA construct. Furthermore, the tests demonstrated that the packaging of either of these sequences resulted in very stable TMV-like particles. Analysis was carried out in duplicate and after multiple freeze-thaw cycles, including dry ice freezing steps and storage for at least 3 months at −20° C.

### Example III

### Test for Successful Generation of “Incoated RNA”

Test principle: free RNA is degraded by treatment with RNase, whereas RNA contained in “Incoated RNA” particles is protected against degradation.

### Example IIIA

### Conventional RNase Protection Assay

The principle of this test procedure has been described previously (Gaddipati, 1988, Nucleic Acids Res 16: 7303-7313).

To test for protection from degradation, pancreatic ribonuclease A was added in a final concentration of 0.1 μg/ml and incubation was allowed to proceed for 15 min at 30° C. Subsequently, proteinase K was added in a final concentration of 1 mg/ml and incubation is continued for an additional 30 min at 30° C. Then Ribonucleoside Vanadyl Complex and SDS were added to a final concentration of 1%, followed by incubation for 10 min at 37° C. The addition of Tris-HCl pH 9.5 (final concentration of 100 mM) was followed by incubation for 10 min at 65° C. Then formamide buffer was added (final concentration of 24% Formamide+0.005% SDS+0.005% bromophenol blue+0.00025% Xylene Cyanol+0.25 mM EDTA), followed by incubation for 15 min at 65° C. Then the sample was loaded on a native agarose (2% agarose in 1×TBE, 0.5 μg/ml EtBr) and electrophoresis was performed at 5 V/cm for 90 min. After de-staining in water for at least 30 min an image is taken on a transilluminator.

### Example IIIB

### RNase Protection Assay Combined with Quantitative RT-PCR

### a) Principle of the Test Procedure

To test for protection from degradation, diluted Incoated RNA particles (about 10⁸ copies/μl) were challenged with heat-labile RNase I (0.01 U/μl) and incubation for 120 min at 37° C. Subsequently, RNA was isolated with RNeasy Minikit. The following control reactions were performed: (i) incubations without RNase, (ii) Incoated RNA particles were destroyed before incubation with RNase. The quantity of isolated WT-RNA was determined in a two-step RT-PCR assay: cDNA synthesis was followed by real-time PCR with a RotorGene 3000 instrument.

### b) Design of Primers and Probes for RT-PCR Assays

The target sequence is the universal 5′-leader sequence (78 bp) and primer and probe sequences were:

5-UNI-F
5′-gAgACgAATTgggCCCTCT
5-UNI-R
5′-AgggCgAATTCTgCAgATATCC

Detection of the resulting PCR product (72 bp) was carried out using the TaqMan type probe 5-Uni-TM 5′-FAM-ggatatctgcagaattcgccct-BBQ.
This approach has the advantage that the RT-PCR target sequence was located within the universal 5′-terminal sequence. Accordingly, these quantitative assays can be used for all Incoated RNA constructs and they provide two test results:
   (i) Demonstration of protection against degradation by RNase.
   (ii) Determination of the IVT-RNA copy numbers in Incoated RNA preparations.
      c) cDNA Synthesis
Free IVT-RNA resulting from the different treatments was used for cDNA synthesis with primer 5-UNI-R. All reagents were obtained from Bioline GmbH (Luckenwalde), primers and probes from TIB MOLBIOL GmbH (Berlin): 3 μl RNA sample+2 μl 10×RT-buffer+2 μl 2 mM dNTPs+1 μl Primer 5-UNI-R (10 pmole/μl)+0.5 μl reverse transcriptase enzyme were mixed, incubation for 1 h at 37° C. was followed by enzyme inaction for 10 min at 72° C., then the samples were put on ice.

### d) Real-Time PCR Assay

For the following quantitative real-time PCR, 5 μl of cDNA were mixed with 15 μl master-mix, containing 10 μl 2× Immomix (Bioline GmbH)+1 μl primer 5-UNI-F (10 pmoles)+1 μl primer 5-UNI-R (10 pmoles)+1 μl probe 5-Uni-TM (5 pmoles)+5′-FAM-ggatatctgcagaattcgccct-BBQ+2 μl water.
Real-time PCR was performed with a RotorGene 3000 instrument (Qiagen GmbH).
Reaction parameters are shown in this table 1, example results are shown in table 2.

TABLE 1

| Reaction step | Temperature | Time |
|---|---|---|
| Denaturation/Enzyme activation | 95° C. | 10 min |
| Amplification | 95° C. | 20 sec |
| for 45 cycles | 60° C. | 20 sec with acquiring |
| | | to Cycling A (FAM) |
| | 72° C. | 20 sec |

TABLE 2

| | | Relative RNA | |
|---|---|---|---|
| | | amount, | |
| | | determined | |
| | | by RT-PCR | |
| | RNase I | (average values | |
| Description | (U/assay) | of 6 reactions) | Conclusion |
| (a) Particles were incubatedfor 120 | 0 | 100% ± 30% | reference value for RNA |
| min at 37° C., w/o RNase, followed | | | contents in the samples |
| by RNA isolation with RNeasy kit | | | |
| (b) Particles were challenged by | 0.1 U/assay | 90% ± 15% | IVT-RNA in intact |
| incubation for 120 min at 37° C. | | | particles was protected; |
| with RNase I, followed by RNA | | | protection factor of |
| isolation with RNeasy kit | | | about 9000, compared |
| | | | with reaction (c) |
| (c) Particles were destroyed before | 0.1 U/assay | 0.01% ± 0.005% | Compared with reaction |
| RNase-treatment by a | | | (b), about 99.99% of |
| first heating step with 10 min at | | | unprotected IVT-RNA |
| 95° C., followed by | | | was degraded |
| incubation for 120 min at 37° C., | | | |
| with RNase I, followed by RNA | | | |
| isolation with RNeasy kit | | | |

Conclusions:
As these results show, IVT-RNA becomes accessible to degradation by RNase after destruction of the particles by heating.
If particles are intact, IVT-RNA is protected against degradation by RNase.
Advantages of IIB versus IIIA: very low amounts of Incoated RNA particles are required, the procedure is easier, involves fewer steps, is faster and results in quantitative data.

### e) Application of Quantitative RT-PCR for Copy Number Determination

The assay was performed as described above, using a dilution series of cDNAs with copy numbers in the range of 10⁸ copies/0 to 10³ copies/0.
The resulting standard curve is shown in FIG. 2, and represented by the following equations:

concentration=10exp(−0,298×CT+11,929) Standard Curve (1)

CT=−3,359×log(concentration)+40,071 Standard Curve (2)

### Example IV

### Stability of Incoated RNA Against Degradation by RNase

Stability tests were performed with Incoated RNA particles (about 10⁸ copies/μl) and incubations with two concentrations of heat-labile RNase I (3 U/assay and 0.3 U/assay) at 37° C. with incubation times between of 120 min and 20 h.

Subsequently, RNA was isolated with RNeasy Minikit. The quantity of isolated IVT-RNA was determined in a two-step RT-PCR assay: cDNA synthesis was followed by real-time PCR with a RotorGene 3000 instrument. All procedures were performed as described above (Example IIIB).

The results are illustrated in FIG. 3 and show that Incoated RNA is stable in the presence of RNase and thus protected from the activity of this enzyme.

### Example V

### Stability of Incoated RNA after Freezing and Thawing

Incoated RNAs were analyzed after one freeze-thaw cycle (1× freeze), i.e. o/n with thaw+assay next day, or after two freeze-thaw cycles (2× freeze), i.e. o/n with thaw+refreeze o/n with thaw and assay next day. Freezing was performed at −20° C. or with dry ice.

The stability of the Incoated RNA particles was observed based on variations in the Ct values after RNase treatment. RT-PCR assays were carried out as described before. For example, if the Incoated RNA particles remain intact, i.e. are substantially unaffected in the treatments, the variations in Ct values do not exceed ±0.5.

### Results

Results of the freeze/thaw stability tests are shown in the below table:

| | Stability of Incoated RNA particles | | | |
|---|---|---|---|---|
| | Ct values after RNase treatment | | | |
| | 1x −20° C. | 2x −20° C. | 1x Dry ice | 2x Dry ice |
| Replica 1 | 17.53 | 17.21 | 17.47 | 17.83 |
| Replica 2 | 17.47 | 17.43 | 17.35 | 17.72 |

It can be seen from the above results that the Ct values in the RT-PCR assays did not change by more than ±0.5 after the freeze-thaw and RNAse-treatments. Accordingly, the Incoated RNA particles are stable during these treatments.

### Example VI

### Stability of Incoated RNA after Long Storage Times at −20° C.

Three aliquots WI, WII and WIII comprising Incoated RNA particles were prepared and split into 5 test samples each. All samples were frozen and stored at −20° C. until the specified test day: samples were then thawed and analyzed by RNase treatment and RT PCR as described above.

### Results:

| | Ct value with RNase after storage at −20° C. for | | | | | Mean for |
|---|---|---|---|---|---|---|
| | 1 day | 22 days | 64 days | 85 days | 141 days | all assays |
| Sample | 18.12 | 18.42 | 18.26 | 18.44 | 18.17 | |
| W I | 18.27 | 18.61 | 18.12 | 18.31 | 18.04 | |
| Sample | 18.34 | 18.09 | 18.27 | 18.08 | 18.28 | |
| W II | 18.47 | 17.80 | 18.42 | 18.08 | 18.17 | |
| Sample | 17.98 | 18.13 | 18.47 | 18.36 | 18.28 | |
| W III | 18.14 | 18.15 | 18.38 | 18.32 | 18.37 | |
| Mean | 18.22 | 18.20 | 18.32 | 18.27 | 18.22 | 18.25 ± 0.5 |

### Conclusions

Incoated RNA particles are stable at −20° C. After 141 days (20 weeks), protection against RNase is maintained, and Ct values remain in the range of 18±0.5.

### Example VII

### Stability of Incoated RNA Under “Accelerated Aging” at +37° C.

Accelerated Aging is an artificial procedure for establishing the lifespan or shelf life of a product in an expedited manner. Data obtained are based on conditions that simulate the effects of aging on the materials. A product can be released to market based upon successful Accelerated Aging test results that simulates the period claimed for product expiration date (1 year, 2 years, etc). The accelerated aging standards for medical devices used here are ASTM F1980-07 (2011).

Accelerated Aging calculation is based on an Arrhenius' equation which essentially states that a 10° C. increase in temperature doubles the rate of a chemical reaction. The following equations are used:
Step 1: AAR=Q10^{(AAT-AT)/10)}
Step 2: AATD=DRTA/AAR

Wherein AAR: Accerlerated Aging Rate; AATD: Accelerated Aging Time Duration; DRTA: Desired Real Time Aging; AAT Accelerated Aging Temperature; AT: Ambient Temperature; Q10: Accelerated Aging Factor (with Q10=2 being industry standard and Q10=1.8 being the more conservative option). This means that four variables are used in calculating the accelerated aging test duration: Test Temperature (° C.), Storage Temperature (° C.), Q10 (Reaction Rate Factor), a conservative number for Q10 is 2 for medical devices and the real-time equivalent, i.e. days.

The AAR for 37° C. (test temperature) and −20° C. (storage temperature can be calculated for both Q10 values:

Q10=2.0=>AAR=51.98

Q10=1.8=>AAR=28.51

### Test Design and Results

Three aliquots WI, WII and WIII were prepared from Incoated RNA and split into 5 test samples. All samples were incubated at 37° C. (incubator oven) until the samples were analyzed on the specified test day. Analysis was as described before by incubation with RNase and RT PCR detection of stable particles.

| | 1 day | 22 days | 43 days |
|---|---|---|---|
| Sample W I | 17.82 | 18.18 | 19.27 |
| | 17.65 | 18.22 | 18.65 |
| Sample W II | 18.30 | 18.44 | 19.60 |
| | 18.11 | 18.29 | 19.74 |
| Sample W III | 17.73 | 17.99 | 18.56 |
| | 18.41 | 18.34 | 18.79 |
| Mean | 18.00 | 18.25 | 19.10 |

### Conclusions

It can be seen from the above, i.e. based on the variation of the Ct values, that the particles are stable at 37° C. for at least about 22 days. Only after 43 days, increased variability and increased Ct values were observed. The increase of 1 in the Ct values indicates that about 50% of the particles remained intact.

With a minimum particle stability of 21 days, the calculated stability at the storage temperature of −20° C. is
1091 days or about 3 years for Q10=2; and
599 days or about 20 months for Q10=1.8.

### Example VIII

### Testing Different TMV OAS Sequences

It had been reported in the art by Turner et al. (1988, J Mol Biol, 203:531-547) that a minimal sequence of 75 nt from the TMV would be sufficient for packaging. Other groups used a TMV OAS having a length of more than 400 nt (Sleat et al., 1986, Virology, 155:299-308). However, it was found that many heterologous RNAs cannot be packaged with these OAS, thus, preventing the efficient use of TMV-like particles in an industrial setting. Further, it was found that in cases in which VLPs assembly took place, the resulting particles were not suitable for storage and use in experiments because they were unstable.

Therefore, several OAS lengths were tested for their assembly efficiency and for the stability of the resulting constructs. In particular, three OAS variations were analyzed having a length of 127, 234 and 593 nucleotides, respectively. All OAS comprised the minimal 75 nt long OAS sequence published by Turner (SEQ ID NO:9, positions 5444-5518 in TMV genome). The tested OAS were termed as follows:

Neu3-1: 127 nt, SEQ ID NO:1, positions 5420-5546 in TMV genome;
Neu3-2: 234 nt, SEQ ID NO:2, positions 5313-5546 in TMV genome; and
Neu3-3: 593 nt, SEQ ID NO:10, positions 5100-5692 in TMV genome.

The constructs further comprised a heterologous target sequence of interest.

It was found that VLP assembly with the Neu3-1 and Neu3-3 OAS constructs was inefficient. After analysis in the above described RNase assay and subsequent RT-PCT analysis no RNA was detected using these constructs. In contrast, assembly with the Neu3-2 construct was very efficient. RNA was efficiently protected in particles that comprised this OAS sequence.

Moreover, it was also found, as already indicated in the previous examples that use of the 234 nt OAS of Neu3-2 provides particularly stable particles that allow storage, shipping and, thus, use of the particles in an industrial setting.

Surprisingly, it was since also observed that the Neu3-2 OAS sequence is suitable as a universal origin of assembly sequence for various RNA sequences. To date, any tested heterologous RNA sequence was successfully packaged with this OAS sequence, including ribosomal RNAs.

### REFERENCES

U.S. Pat. No. 5,677,124;
US 2002/0192689;
Villanova et al., 2007, J Clin Microbiol, p. 3555-63;
Sleat et al., 1986, Virology, 155:299-308,
Turner et al., 1988, J Mol Biol, 203:531-547;
U.S. Pat. No. 5,256,555
Sambrook et al., 2000, Molecular Cloning, A laboratory manual, 3^{rd} ed., Cold Spring Harbour Laboratory;
Kadri et al., (2013, J Virol Methods, 189: 328-340;
Butler, 1984, J Gen Virol, 65:253-279;
Durham, 1972, J Mol Biol, 67:289-305,
Wu et al., 2010, ACS Nano, 4:4531-8;
Mueller et al., 2010, J Virol Methods, 166: 77-85;
Wu et al., 2010, ACS Nano, 4:4531-8

1. Method for the detection of RNA in a sample, comprising
(a) mixing a specific amount of a rod-shaped virus-like particle with a sample, wherein the rod-shaped virus-like particle comprises a ribonucleic acid molecule and a viral coat, wherein:
(i) the ribonucleic acid molecule comprises an origin-of-assembly sequence of a rod-shaped RNA virus and a heterologous sequence; and
(ii) the viral coat comprises at least one type of coat protein of the rod-shaped RNA virus;
(b) isolating RNA from the sample; and
(c) detecting RNA comprising the heterologous sequence.

2. The method for the detection of RNA in a sample according to claim 1, wherein the origin-of-assembly sequence has a length of from 150 to 300 nucleotides.

3. The method for the detection of RNA in a sample according to claim 1, wherein the origin-of-assembly is a sequence having a length of up to 300 nucleotides comprising:
(A) the sequence of SEQ ID NO:2; or
(B) a sequence having at least 85% identity to the sequence of SEQ ID NO:2; or
(C) a fragment of (A) or (B), wherein the fragment has a length of at least 150 nucleotides.

4. The method for the detection of RNA in a sample according to claim 1, wherein the origin-of-assembly sequence is directly at the 3′-end of the ribonucleic acid molecule.

5. The method for the detection of RNA in a sample according to claim 1, comprising the quantitative detection of RNA.

6. The method for the detection of RNA in a sample according to claim 1, further comprising the detection of a second RNA of interest in the sample.

7. The method for the detection of RNA in a sample according to claim 1, further comprising a step, wherein the ratio between RNA of interest and the RNA with the heterologous sequence is determined.

8. The method for the detection of RNA in a sample according to claim 5, wherein the RNA of interest and the RNA with the heterologous sequence are detected by the same detection method.

9. The method for the detection of RNA in a sample according to claim 1, wherein the detection method comprises reverse transcription, polymerase chain reaction (PCR), other nucleic acid amplification methods, such as nucleic acid sequence based amplification (NASBA), Northern blot, and/or branched DNA.

10. The method for the detection of RNA in a sample according to claim 1, wherein the rod-shaped virus is Tobacco Mosaic Virus (WV).

11. Use of a rod-shaped virus-like particle as an internal RNA standard or as a positive control for RNA degradation and/or RNA quantification, wherein the rod-shaped virus-like particle comprises a ribonucleic acid molecule and a viral coat, wherein:
(a) the ribonucleic acid molecule comprises an origin-of-assembly sequence of a rod-shaped RNA virus and a heterologous sequence; and
(b) the viral coat comprises at least one type of coat protein of the rod-shaped RNA virus.

12. Rod-shaped virus-like particle comprising a ribonucleic acid molecule and a viral coat, wherein:
(a) the ribonucleic acid molecule comprises an origin-of-assembly sequence of a rod-shaped RNA virus and a heterologous sequence, wherein the origin-of-assembly sequence has a length of from 150 to 300 nucleotides; and
(n) the viral coat comprises at least one type of coat protein of the rod-shaped RNA virus.

13. Rod-shaped virus-like particle according to claim 12, wherein the origin-of-assembly is a sequence having a length of up to 300 nucleotides comprising:
(A) the sequence of SEQ ID NO:2: or
(B) a sequence having at least 85% identity to the sequence of SEQ ID NO:2; or
(C) a fragment of (A) or (B), wherein the fragment has a length of at least 150 nucleotides.

14. The rod-shaped virus-like particle according to claim 12, wherein the origin-of-assembly sequence is directly at the 3′-end of the ribonucleic acid molecule.

15. The rod-shaped virus-like particle according to claim 12, wherein the rod-shaped virus is Tobacco Mosaic Virus (TMV).


SEQUENCE LISTING (10 sequences)

SEQ ID NO: 1 - RNA, 127 nt, Tobacco mosaic virus
guuugagagagaagauuacaaacgugagagacggagggcccauggaacuuacagaagaagucguugaugaguucauggaagaugucccuaugucgaucaggcuugcaaaguuucgaucucgaaccgg

SEQ ID NO: 2 - RNA, 234 nt, Tobacco mosaic virus
gcaaguuuuaguuaauauuagaaaugugaagaugucagcggguuucuguccgcuuucucuggaguuugugucgguguguauuguuuauagaaauaauauaaaauuagguuugagagagaagauuacaaacgugagagacggagggcccauggaacuuacagaagaagucguugaugaguucauggaagaugucccuaugucgaucaggcuugcaaaguuucgaucucgaaccgg

SEQ ID NO: 3 - RNA, 78 nt, artificial
  universal 5′-leader sequence
gggagacgaauugggcccucuagaugcaugcucgagcggccgccagugugauggauaucugcagaauucgcccuuauu

SEQ ID NO: 4 - RNA, 78 nt, artificial
  complementary sequence to the 5′-leader
aauaagggcgaauucugcagauauccaucacacuggcggccgcucgagcaugcaucuagagggcccaauucgucuccc

SEQ ID NO: 5 - protein, 159 aa, Tobacco mosaic virus
MSYSITTPSQFVFLSSAWADPIELINLCTNALGNQFQTQQARTVVQRQFSEVWKPSPQVTVRFPDSDFKVYRYNAVLDPLVTALLGAFDTRNRIIEVENQANPTTAETLDATRRVDDATVAIRSAINNLIVELIRGTGSYNRSSFESSSGLVWTSGPAT

SEQ ID NO: 6 - RNA, 6395 nt, Tobacco mosaic virus
guauuuuuacaacaauuaccaacaacaacaaacaacaaacaacauuacaauuacuauuuacaauuacaauggcauacacacagacagcuaccacaucagcuuugcuggacacuguccgaggaaacaacuccuuggucaaugaucuagcaaagcgucgucuuuacgacacagcgguugaagaguuuaacgcucgugaccgcaggcccaaggugaacuuuucaaaaguaauaagcgaggagcagacgcuuauugcuacccgggcguauccagaauuccaaauuacauuuuauaacacgcaaaaugccgugcauucgcuugcagguggauugcgaucuuuagaacuggaauaucugaugaugcaaauucccuacggaucauugacuuaugacauaggcgggaauuuugcaucgcaucuguucaagggacgagcauauguacacugcugcaugcccaaccuggacguucgagacaucaugcggcacgaaggccagaaagacaguauugaacuauaccuuucuaggcuagagagaggggggaaaacaguccccaacuuccaaaaggaagcauuugacagauacgcagaaauuccugaagacgcugucugucacaauacuuuccagacaaugcgacaucagccgaugcagcaaucaggcagaguguaugccauugcgcuacacagcauauaugacauaccagccgaugaguucggggcggcacucuugaggaaaaauguccauacgugcuaugccgcuuuccacuucucugagaaccugcuucuugaagauucauacgucaauuuggacgaaaucaacgcguguuuuucgcgcgauggagacaaguugaccuuuucuuuugcaucagagaguacucuuaauuauugucauaguuauucuaauauucuuaaguaugugugcaaaacuuacuucccggccucuaauagagagguuuacaugaaggaguuuuuagucaccagaguuaauaccugguuuuguaaguuuucuagaauagauacuuuucuuuuguacaaagguguggcccauaaaaguguagauagugagcaguuuuauacugcaauggaagacgcauggcauuacaaaaagacucuugcaaugugcaacagcgagagaauccuccuugaggauucaucaucagucaauuacugguuucccaaaaugagggauauggucaucguaccauuauucgacauuucuuuggagacuaguaagaggacgcgcaaggaagucuuaguguccaaggauuucguguuuacagugcuuaaccacauucgaacauaccaggcgaaagcucuuacauacgcaaauguuuuguccuuugucgaaucgauucgaucgaggguaaucauuaacggugugacagcgagguccgaaugggauguggacaaaucuuuguuacaauccuuguccaugacguuuuaccugcauacuaagcuugccguucuaaaggaugacuuacugauuagcaaguuuagucucgguucgaaaacggugugccagcaugugugggaugagauuucgcuggcguuugggaacgcauuucccuccgugaaagagaggcucuugaacaggaaacuuaucagaguggcaggcgacgcauuagagaucagggugccugaucuauaugugaccuuccacgacagauuagugacugaguacaaggccucuguggacaugccugcgcuugacauuaggaagaagauggaagaaacggaagugauguacaaugcacuuucagaguuaucgguguuaagggagucugacaaauucgauguugauguuuuuucccagaugugccaaucuuuggaaguugacccaaugacggcagcgaagguuauagucgcggucaugagcaaugagagcggucugacucucacauuugaacgaccuacugaggcgaauguugcgcuagcuuuacaggaucaagagaaggcuucagaaggugcuuugguaguuaccucaagagaaguugaagaaccguccaugaaggguucgauggccagaggagaguuacaauuagcuggucuugcuggagaucauccggagucguccuauucuaagaacgaggagauagagucuuuagagcaguuucauauggcaacggcagauucguuaauucguaagcagaugagcucgauuguguacacggguccgauuaaaguucagcaaaugaaaaacuuuaucgauagccugguagcaucacuaucugcugcggugucgaaucucgucaagauccucaaagauacagcugcuauugaccuugaaacccgucaaaaguuuggagucuuggauguugcaucuaggaagugguuaaucaaaccaacggccaagagucaugcaugggguguuguugaaacccacgcgaggaaguaucauguggcgcuuuuggaauaugaugagcaggguguggugacaugcgaugauuggagaagaguagcugucagcucugagucuguuguuuauuccgacauggcgaaacucagaacucugcgcagacugcuucgaaacggagaaccgcaugucaguagcgcaaagguuguucuuguggacggaguuccgggcugugggaaaaccaaagaaauucuuuccaggguuaauuuugaugaagaucuaauuuuaguaccugggaagcaagccgcggaaaugaucagaagacgugcgaauuccucagggauuauuguggccacgaaggacaacguuaaaaccguugauucuuucaugaugaauuuugggaaaagcacacgcugucaguucaagagguuauucauugaugaaggguugauguugcauacugguuguguuaauuuucuuguggcgaugucauugugcgaaauugcauauguuuacggagacacacagcagauuccauacaucaauagaguuucaggauucccguaccccgcccauuuugccaaauuggaaguugacgagguggagacacgcagaacuacucuccguuguccagccgaugucacacauuaucugaacaggagauaugagggcuuugucaugagcacuucuucgguuaaaaagucuguuucgcaggagauggucggcggagccgccgugaucaauccgaucucaaaacccuugcauggcaagauccugacuuuuacccaaucggauaaagaagcucugcuuucaagaggguauucagauguucacacugugcaugaagugcaaggcgagacauacucugauguuucacuaguuagguuaaccccuacaccagucuccaucauugcaggagacagcccacauguuuuggucgcauugucaaggcacaccuguucgcucaaguacuacacuguuguuauggauccuuuaguuaguaucauuagagaucuagagaaacuuagcucguacuuguuagauauguauaaggucgaugcaggaacacaauagcaauuacagauugacucgguguucaaagguuccaaucuuuuuguugcagcgccaaagacuggugauauuucugauaugcaguuuuacuaugauaagugucucccaggcaacagcaccaugaugaauaauuuugaugcuguuaccaugagguugacugacauuucauugaaugucaaagauugcauauuggauaugucuaagucuguugcugcgccuaaggaucaaaucaaaccacuaauaccuaugguacgaacggcggcagaaaugccacgccagacuggacuauuggaaaauuuaguggcgaugauuaaaaggaacuuuaacgcacccgaguugucuggcaucauugauauugaaaauacugcaucuuuaguuguagauaaguuuuuugauaguuauuugcuuaaagaaaaaagaaaaccaaauaaaaauguuucuuuguucaguagagagucucucaauagaugguuagaaaagcaggaacagguaacaauaggccagcucgcagauuuugauuuuguagauuugccagcaguugaucaguacagacacaugauuaaagcacaacccaagcaaaaauuggacacuucaauccaaacggaguacccggcuuugcagacgauuguguaccauucaaaaaagaucaaugcaauauuuggcccguuguuuagugagcuuacuaggcaauuacuggacaguguugauucgagcagauuuuuguuuuucacaagaaagacaccagcgcagauugaggauuucuucggagaucucgacagucaugugccgauggaugucuuggagcuggauauaucaaaauacgacaaaucucagaaugaauuccacugugcaguagaauacgagaucuggcgaagauuggguuuugaagacuucuugggagaaguuuggaaacaagggcauagaaagaccacccucaaggauuauaccgcagguauaaaaacuugcaucugguaucaaagaaagagcggggacgucacgacguucauuggaaacacugugaucauugcugcauguuuggccucgaugcuuccgauggagaaaauaaucaaaggagccuuuugcggugacgauagucugcuguacuuuccaaaggguugugaguuuccggaugugcaacacuccgcgaaucuuauguggaauuuugaagcaaaacuguuuaaaaaacaguauggauacuuuugcggaagauauguaauacaucacgacagaggaugcauuguguauuacgauccccuaaaguugaucucgaaacuuggugcuaaacacaucaaggauugggaacacuuggaggaguucagaaggucucuuugugauguugcuguuucguugaacaauugugcguauuacacacaguuggacgacgcuguaugggagguucauaagaccgccccuccagguucguuuguuuauaaaagucuggugaaguauuugucugauaaaguucuuuuuagaaguuuguuuauagauggcucuaguuguuaaaggaaaagugaauaucaaugaguuuaucgaccugacaaaaauggagaagaucuuaccgucgauguuuaccccuguaaagaguguuauguguuccaaaguugauaaaauaaugguucaugagaaugagucauugucagaggugaaccuucuuaaaggaguuaagcuuauugauaguggauacgucuguuuagccgguuuggucgucacgggcgaguggaacuugccugacaauugcagaggaggugugagcgugugucugguggacaaaaggauggaaagagccgacgaggccacucucggaucuuacuacacagcagcugcaaagaaaagauuucaguucaaggucguucccaauuaugcuauaaccacccaggacgcgaugaaaaacgucuggcaaguuuuaguuaauauuagaaaugugaagaugucagcggguuucuguccgcuuucucuggaguuugugucgguguguauuguuuauagaaauaauauaaaauuagguuugagagagaagauuacaaacgugagagacggagggcccauggaacuuacagaagaagucguugaugaguucauggaagaugucccuaugucgaucaggcuugcaaaguuucgaucucgaaccggaaaaaagagugauguccgcaaagggaaaaauaguaguaaugaucggucagugccgaacaagaacuauagaaauguuaaggauuuuggaggaaugaguuuuaaaaagaauaauuuaaucgaugaugauucggaggcuacugucgccgaaucggauucguuuuaaauaugucuuacaguaucacuacuccaucucaguucguguucuugucaucagcgugggccgacccaauagaguuaauuaauuuauguacuaaugccuuaggaaaucaguuucaaacacaacaagcucgaacugucguucaaagacaauucagugagguguggaaaccuucaccacaaguaacuguuagguucccugacagugacuuuaagguguacagguacaaugcgguauuagacccgcuagucacagcacuguuaggugcauucgacacuagaaauagaauaauagaaguugaaaaucaggcgaaccccacgacugccgaaacguuagaugcuacucguagaguagacgacgcaacgguggccauaaggagcgcgauaaauaauuuaauaguagaauugaucagaggaaccggaucuuauaaucggagcucuuucgagagcucuucugguuugguuuggaccucugguccugcaacuugagguagucaagaugcauaauaaauaacggauuguguccguaaucacacguggugcguacgauaacgcauaguguuuuucccuccacuuaaaucgaaggguugugucuuggaucgcgcgggucaaauguauaugguucauauacauccgcaggcacguaauaaagcgagggguucgaauccccccguuacccccgguaggggccca

SEQ ID NO: 7 - DNA, 914 bp, artificial
  Template for Incoated Domain I 16S rRNA
gaatttaatacgactcactatagggagacgaattgggccctctagatgcatgctcgagcggccgccagtgtgatggatatctgcagaattcgcccttattaaattgaagagtttgatcatggctcagattgaacgctggcggcaggcctaacacatgcaagtcgaacggtaacaggaagcagcttgctgcttcgctgacgagtggcggacgggtgagtaatgtctgggaagctgcctgatggagggggataactactggaaacggtagctaataccgcataatgtcgcaagaccaaagagggggaccttcgggcctcttgccatcggatgtgcccagatgggattagcttgttggtggggtaacggctcaccaaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaagttttagttaatattagaaatgtgaagatgtcagcgggtttctgtccgctttctctggagtttgtgtcggtgtgtattgtttatagaaataatataaaattaggtttgagagagaagattacaaacgtgagagacggagggcccatggaacttacagaagaagtcgttgatgagttcatggaagatgtccctatgtcgatcaggcttgcaaagtttcgatctcgaaccgg

SEQ ID NO: 8 - DNA, 1876 bp, artificial
  Template for incoated 16S rRNA complete
gaatttaatacgactcactatagggagacgaattgggccctctagatgcatgctcgagcggccgccagtgtgatggatatctgcagaattcgcccttattaaattgaagagtttgatcatggctcagattgaacgctggcggcaggcctaacacatgcaagtcgaacggtaacaggaagcagcttgctgcttcgctgacgagtggcggacgggtgagtaatgtctgggaagctgcctgatggagggggataactactggaaacggtagctaataccgcataatgtcgcaagaccaaagagggggaccttcgggcctcttgccatcggatgtgcccagatgggattagcttgttggtggggtaacggctcaccaaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctccttagcaagttttagttaatattagaaatgtgaagatgtcagcgggtttctgtccgctttctctggagtttgtgtcggtgtgtattgtttatagaaataatataaaattaggtttgagagagaagattacaaacgtgagagacggagggcccatggaacttacagaagaagtcgttgatgagttcatggaagatgtccctatgtcgatcaggcttgcaaagtttcgatctcgaaccgg

SEQ ID NO: 9 - RNA, 75 nt, Tobacco mosaic virus
ugagagacggagggcccauggaacuuacagaagaagucguugaugaguucauggaagaugucccuaugucgauca

SEQ ID NO: 10 - RNA, 593 nt, Tobacco mosaic virus
cgucuguuuagccgguuuggucgucacgggcgaguggaacuugccugacaauugcagaggaggugugagcgugugucugguggacaaaaggauggaaagagccgacgaggccacucucggaucuuacuacacagcagcugcaaagaaaagauuucaguucaaggucguucccaauuaugcuauaaccacccaggacgcgaugaaaaacgucuggcaaguuuuaguuaauauuagaaaugugaagaugucagcggguuucuguccgcuuucucuggaguuugugucgguguguauuguuuauagaaauaauauaaaauuagguuugagagagaagauuacaaacgugagagacggagggcccauggaacuuacagaagaagucguugaugaguucauggaagaugucccuaugucgaucaggcuugcaaaguuucgaucucgaaccggaaaaaagagugauguccgcaaagggaaaaauaguaguaaugaucggucagugccgaacaagaacuauagaaauguuaaggauuuuggaggaaugaguuuuaaaaagaauaauuuaaucgaugaugauucggaggcuacugucgccg